(19) United States
(12) Patent Application Publication
(10) Pub. No.: US 2021/0204550 A1
(43) Pub. Date: Jul. 8, 2021

(54) STABILIZED FUNGICIDAL COMPOSITION

(71) Applicant: Bayer CropScience LP, St. Louis, MO (US)

(72) Inventors: Andreas GÖRTZ, Dormagen (DE); Chad KIMMELSHUE, Ames, IA (US); Yan LI, Citrus Heights, CA (US); Colleen S. TAYLOR, Folsom, CA (US); Cecilia R. WILSON, West Sacramento, CA (US); Kristina FINSETH, Sacramento, CA (US)

(21) Appl. No.: 17/055,923

(22) PCT Filed: May 14, 2019
(86) PCT No.: PCT/US2019/032273

§ 371 Date: Nov. 16, 2020

Related U.S. Application Data
(60) Provisional application No. 62/672,969, filed on May 17, 2018.

(51) Int. Cl. A01N 63/25; A01N 25/22

(52) U.S. Cl. CPC A01N 63/25; A01N 25/22

(57) ABSTRACT

The present invention relates to a stabilized fungicidal composition comprising: a) a fusaricidin-producing Paenibacillus sp. strain; and b) a stabilizing agent selected from the group consisting of an amine, a quaternary ammonium compound, a salt of phosphate or citrate, a short chain polyol having from 2 to 10 carbon atoms, a urea, and combinations thereof. The present invention also provides agriculturally acceptable stable aqueous formulations comprising a fusaricidin-producing Paenibacillus sp. strain and a stabilizing agent and methods of controlling disease in plants with the fungicidal compositions and aqueous formulations.

### CROSS REFERENCE TO RELATED APPLICATIONS

This application claims priority to U.S. Provisional Patent Application No. 62/672,969, filed May 17, 2018, the contents of which are incorporated herein by reference in their entirety.

### TECHNICAL FIELD

The present invention relates to formulations of bacterial strains and methods of their use to control plant diseases. In particular, the present invention is directed to stable fungicidal compositions and formulations of a fusaricidin-producing Paenibacillus sp. strain.

### BACKGROUND

Fungicides have myriad uses, including for crop protection; as food, feed, and cosmetics preservatives; and as therapeutic agents for both human and veterinary applications. Crop yield reduction, foodborne diseases and fungal infections of both humans and animals are a problem in both developed and developing countries.

Synthetic insecticides or fungicides often are non-specific and therefore can act on organisms other than the target ones, including other naturally occurring beneficial organisms. Because of their chemical nature, they may also be toxic and non-biodegradable. Consumers worldwide are increasingly conscious of the potential environmental and health problems associated with the residuals of chemicals, particularly in food products. This has resulted in growing consumer pressure to reduce the use or at least the quantity of chemical (i.e., synthetic) pesticides. Thus, there is a need to manage food chain requirements while still allowing effective pest control.

A further problem arising with the use of synthetic insecticides or fungicides is that the repeated and exclusive application of an insecticide or fungicide often leads to selection of resistant pathogenic microorganisms. Normally, such strains are also cross-resistant against other active ingredients having the same mode of action. An effective control of the pathogens with said active compounds is then not possible any longer. However, active ingredients having new mechanisms of action are difficult and expensive to develop.

The risk of resistance development in pathogen populations as well as environmental and human health concerns have fostered interest in identifying alternatives to synthetic insecticides and fungicides for managing plant diseases. The use of biological control agents is one alternative.

Paenibacillus-based biological control agents can provide a valuable tool to control a spectrum of plant diseases while reducing the risk of pathogen resistance. Fusaricidins and other antifungal compounds are produced during fermentation of Paenibacillus sp. strains. However, these antifungal compounds are susceptible to degradation during storage and shipment of the Paenibacillus-based biological control agents. Elevated temperatures common during growing seasons contribute to the degradation of these antifungal compounds. There is a need to identify stabilized compositions and formulations of Paenibacillus-based biological control agents that will maintain their activity to control plant disease over extended periods of time and at elevated temperatures.

### SUMMARY

The present invention is based on the identification of stabilizing agents that inhibit the degradation of the antifungal compounds produced by a Paenibacillus sp. strain. These stabilizing agents preserve the antifungal activity of the formulated Paenibacillus sp. strain and reduce the breakdown of fusaricidins and fusaricidin-like compounds.

In certain embodiments, the present invention is directed to a stabilized fungicidal composition comprising: a) a fusaricidin-producing Paenibacillus sp. strain; and b) a stabilizing agent selected from the group consisting of an amine, a quaternary ammonium compound, a salt of phosphate or citrate, a short chain polyol having from 2 to 10 carbon atoms, a urea, and combinations thereof.

In other embodiments, the present invention is directed to a stabilized fungicidal composition comprising: a) a fusaricidin-producing Paenibacillus sp. strain; and b) a stabilizing agent selected from the group consisting of an amine, a quaternary ammonium compound, a salt of phosphate or citrate, a short chain polyol having from 2 to 10 carbon atoms, a urea, an alkanolamine, an amide, and combinations thereof.

In some embodiments, the present invention is directed to a stabilized fungicidal composition comprising: a) a fusaricidin-producing Paenibacillus sp. strain; and b) a stabilizing agent selected from the group consisting of carbonic diamide (CO(NH₂)₂), guanidine hydrochloride, triethanolamine, betaine hydrochloride, potassium phosphate (dibasic), potassium citrate (tribasic), and a combination thereof.

In certain aspects, the composition or formulation comprises a biologically pure culture of the fusaricidin-producing Paenibacillus sp. strain.

In one embodiment, the fusaricidin-producing Paenibacillus sp. strain is P. agarexedens, P. agaridevorans, P. alginolyticus, P. alkaliterrae, P. alvei, P. amylolyticus, P. anaericanus, P. antarcticus, P. assamensis, P. azoreducens, P. azotofixans, P. barcinonensis, P. borealis, P. brasiliensis, P. brassicae, P. campinasensis, P. chinjuensis, P. chitinolyticus, P. chondroitinus, P. cineris, P. cookie, P. curdlanolyticus, P. daejeonensis, P. dendritiformis, P. durum, P. ehimensis, P. elgii, P. favisporus, P. glucanolyticus, P. glycanilyticus, P. gordonae, P. graminis, P. granivorans, P. hodogayensis, P. illinoisensis, P. jamilae, P. kobensis, P. koleovorans, P. koreensis, P. kribbensis, P. lactis, P. larvae, P. lautus, P. lentimorbus, P. macerans, P. macquariensis, P. massiliensis, P. mendelii, P. motobuensis, P. naphthalenovorans, P. nematophilus, P. nov. spec. epiphyticus, P. odorifer, P. pabuli, P. peoriae, P. phoenicis, P. phyllosphaerae, P. polymyxa, P. polymyxa ssp. polymyxa, P. polymyxa ssp. plantarum, P. popilliae, P. pulvifaciens, P. rhizosphaerae, P. sanguinis, P. stellifer, P. taichungensis, P. terrae, P. thiaminolyticus, P. timonensis, P. tylopili, P. turicensis, P. validus, P. vortex, P. vulneris, P. wynnii or P. xylanilyticus.

In another embodiment, the fusaricidin-producing Paenibacillus sp. strain is Paenibacillus polymyxa, Paenibacillus polymyxa ssp. polymyxa, Paenibacillus polymyxa ssp. plantarum, Paenibacillus nov. spec. epiphyticus, Paenibacillus terrae, Paenibacillus macerans, or Paenibacillus alvei. In yet another embodiment, the fusaricidin-producing Paenibacillus sp. strain is Paenibacillus terrae.

The fungicidal composition can be in any formulation form, particularly a liquid composition, such as an emulsifiable concentrate, suspoemulsion, suspension concentrate, or a solution, such as an aqueous solution.

In some aspects, the weight to weight ratio of the fusaricidin-producing Paenibacillus sp. strain and the stabilizing agent is about 500:1 to about 1:500, about 400:1 to about 1:400, about 300:1 to about 1:300, about 200:1 to about 1:200, about 100:1 to about 1:100, about 75:1 to about 1:75, or about 50:1 to about 1:50. In one embodiment, the weight to weight ratio of the fusaricidin-producing Paenibacillus sp. strain and the stabilizing agent is about 200:1 to about 1:200. In another embodiment, the weight to weight ratio of the fusaricidin-producing Paenibacillus sp. strain and the stabilizing agent is about 50:1 to about 1:50.

In certain aspects, the fungicidal composition further comprises a polar water miscible organic solvent. The polar water miscible organic solvent may be any one of 1,3 butylene glycol, 2-pyrrolidone, acetone, acetonitrile, an aliphatic alcohol, an aliphatic carboxylic acid alkyl ester, cyclohexanone, di- and triglycols, diacetone alcohol, dialkyl ketone, diethylene glycol, diglyme, DMF, DMSO, ethanol, ethyl acetate, formamide, furfuryl alcohol, gamma-butyrolactone, glycerol, glycofurol, a glycol ether, glycol, hexamethylene glycol, isopropanol, methyl ethyl ketone, N-methyl pyrrolidone, pentamethylene glycol, phosphoric acid esters, polyethylene glycol, polyethylene glycols, polyhydroxylated alkanes, propanol, propylene carbonate, propylene glycol, pyrrolidine, pyrrolidine, sulfolane, tetrahydrofuran, tetramethylene glycol, thiodiglycol, triethylene glycol and/or combinations thereof.

In certain aspects, the present invention is directed to a composition wherein the fusaricidin-producing Paenibacillus sp. strain is Paenibacillus sp. strain NRRL B-50972, Paenibacillus sp. strain NRRL B-67129, Paenibacillus sp. strain NRRL B-67304, Paenibacillus sp. strain NRRL B-67306, Paenibacillus sp. strain NRRL B-67615, or a fungicidal mutant strain thereof. The composition may comprise a fermentation product of Paenibacillus sp. strain NRRL B-50972, Paenibacillus sp. strain NRRL B-67129, Paenibacillus sp. strain NRRL B-67304, Paenibacillus sp. strain NRRL B-67306, Paenibacillus sp. strain NRRL B-67615, or a fungicidal mutant strain thereof.

In some embodiments, the fungicidal mutant strain has a genomic sequence with greater than about 90% sequence identity to Paenibacillus sp. strain NRRL B-50972, Paenibacillus sp. strain NRRL B-67129, Paenibacillus sp. strain NRRL B-67304, Paenibacillus sp. strain NRRL B-67306, or Paenibacillus sp. strain NRRL B-67615. In other embodiments, the fungicidal mutant strain has fungicidal activity and/or levels of a fusaricidin that are comparable or better than that of Paenibacillus sp. NRRL B-50972.

In one aspect, the present invention is directed to an agriculturally acceptable stable aqueous formulation comprising: (a) a fusaricidin-producing Paenibacillus sp. strain in an amount of 3% w/w to 90% w/w; (b) a stabilizing agent selected from the group consisting of an amine, a quaternary ammonium compound, a salt of phosphate or citrate, a short chain polyol having from 2 to 10 carbon atoms, a urea, and combinations thereof in an amount of 0.1% w/w to 10% w/w; (c) water; and (d) optionally, a polar water miscible organic solvent in an amount of 2% w/w to 60% w/w.

In another aspect, the present invention is directed to an agriculturally acceptable stable aqueous formulation comprising: (a) a fusaricidin-producing Paenibacillus sp. strain in an amount of 3% w/w to 90% w/w; (b) a stabilizing agent selected from the group consisting of carbonic diamide (CO(NH₂)₂), guanidine hydrochloride, triethanolamine, betaine hydrochloride, potassium phosphate (dibasic), potassium citrate (tribasic), and combinations thereof in an amount of 0.1% w/w to 10% w/w; (c) water; and (d) optionally, a polar water miscible organic solvent in an amount of 2% w/w to 60% w/w.

The amount of fusaricidin-producing Paenibacillus sp. strain employed is preferably about 3% w/w to about 90% w/w of the entire formulation. More preferably, the fusaricidin-producing Paenibacillus sp. strain is present in an amount of about, from 4% to 80% w/w, more preferably 5% w/w to about 75% w/w, and, in some embodiments, from about 10% w/w to about 70% w/w or even about 20% w/w to about 70% w/w.

The amount of stabilizing agent employed is preferably about 0.1% w/w to about 20% w/w of the entire formulation. More preferably, the stabilizing agent is present in an amount of about, from 0.5% to 10% w/w, more preferably 0.5% w/w to about 5% w/w, and, in some embodiments, from about 0.5% w/w to about 2% w/w.

The amount of polar water miscible organic solvent employed is optionally about 2% w/w to about 60% w/w of the entire formulation. Alternatively, the polar water miscible organic solvent is optionally present in an amount of about, from 5% to 50% w/w, more preferably 10% w/w to about 40% w/w, and, in some embodiments, from about 20% w/w to about 40% w/w.

In certain aspects, the present invention is directed to stable dry formulations comprising (a) a fusaricidin-producing Paenibacillus sp. strain; and (b) a stabilizing agent selected from the group consisting of an amine, a quaternary ammonium compound, a salt of phosphate or citrate, a short chain polyol having from 2 to 10 carbon atoms, a urea, and combinations thereof.

In other aspects, the present invention is directed to stable dry formulations comprising (a) a fusaricidin-producing Paenibacillus sp. strain; and (b) a stabilizing agent selected from the group consisting of an amine, a quaternary ammonium compound, a salt of phosphate or citrate, a short chain polyol having from 2 to 10 carbon atoms, a urea, an alkanolamine, an amide, and combinations thereof.

The dry formulation may be any one of a wettable powder, a soluble powder, a dust, a granule, a tablet, a water-soluble and water-dispersible granule, a water-soluble and water-dispersible tablet, and a water-soluble and water-dispersible powder.

In one embodiment, the present invention is directed to an agriculturally acceptable stable formulation comprising: (a) a fusaricidin-producing Paenibacillus sp. strain in an amount of 3% w/w to 90% w/w; (b) a stabilizing agent selected from the group consisting of an amine, a quaternary ammonium compound, a salt of phosphate or citrate, a short chain polyol having from 2 to 10 carbon atoms, a urea, and combinations thereof in an amount of 0.1% w/w to 10% w/w; and (c) other formulation inerts.

In yet another embodiment, the present invention relates to a method of treating a plant to control a disease, wherein the method comprises applying an effective amount of a composition or formulation disclosed herein to the plant, to a part of the plant and/or to a locus of the plant. In certain aspects, the composition comprises a fermentation product of the Paenibacillus sp. strain NRRL B-50972, Paenibacillus sp. strain NRRL B-67129, Paenibacillus sp. strain NRRL B-67304, Paenibacillus sp. strain NRRL B-67306, Paenibacillus sp. strain NRRL B-67615, or a fungicidal mutant strain thereof. In other aspects, the method comprises applying the composition to foliar plant parts. In yet other aspects, the composition is applied at about 1×10¹⁰ to about 1×10¹² colony forming units (CFU) of Paenibacillus sp. strain NRRL B-50972, Paenibacillus sp. strain NRRL B-67129, Paenibacillus sp. strain NRRL B-67304, Paenibacillus sp. strain NRRL B-67306, Paenibacillus sp. strain NRRL B-67615, or a fungicidal mutant strain thereof per hectare. In one embodiment, the composition is applied at about 0.5 kg to about 5 kg fermentation solids per hectare.

In some aspects, the plant disease is caused by a fungus. In other aspects the plant disease is mildew or a rust disease. In one embodiment, the mildew is powdery mildew or downy mildew. In another embodiment, the rust disease is selected from the group consisting of wheat leaf rust, leaf rust of barley, leaf rust of rye, brown leaf rust, crown rust, and stem rust.

In some embodiments, the fungus is selected from the group consisting of Alternaria alternata, Alternaria solani, Botrytis cinerea, Colletotrichum lagenarium, Fusarium culmorum, Phaeosphaeria nodorum, Zymoseptoria tritici, Phytophthora cryptogea, Phytophthora infestans, Pythium ultimum, Magnaporthe oryzae, Thanatephorus cucumeris, Ustilago segetum var. avenae, Uromyces appendiculatus, and Puccinia triticina.

The present invention also relates to the use of the disclosed compositions and formulations for controlling a phytopathogenic organism in useful plants. In certain aspects, the phytopathogenic organism is selected from the group consisting of Alternaria alternata, Alternaria solani, Botrytis cinerea, Colletotrichum lagenarium, Fusarium culmorum, Phaeosphaeria nodorum, Zymoseptoria tritici, Phytophthora cryptogea, Phytophthora infestans, Pythium ultimum, Magnaporthe oryzae, Thanatephorus cucumeris, Ustilago segetum var. avenae, Uromyces appendiculatus, and Puccinia triticina. In other aspects, the phytopathogenic organism is selected from the group consisting of Xanthomonas campestris, Pseudomonas syringae, and Erwinia carotovora.

In yet other aspects, the useful plants are selected from the group consisting of apples, bananas, citrus, kiwi, melons, peaches, pears, pineapple, pome fruit, pomegranate, cabbage, cauliflower, cucumbers, cucurbits, tomatoes, potatoes, wheat, rice and soybeans.

### BRIEF DESCRIPTION OF THE DRAWINGS

FIG. 1 depicts relative fusaricidin levels in liquid formulations of Paenibacillus sp. strain NRRL B-50972 broth concentrate (BC) without any stabilizing agent or mixed with urea, guanidine hydrochloride, triethanolamine, or glycerol ethyl oxylate before and after storage at 54° C. for two weeks.

FIG. 2 depicts relative fusaricidin levels in liquid formulations of Paenibacillus sp. strain NRRL B-50972 BC without any stabilizing agent or mixed with urea, guanidine hydrochloride, triethanolamine, or glycerol ethyl oxylate before and after storage at 40° C. for two, four, and eight weeks.

FIG. 3 depicts relative fusaricidin levels in liquid formulations of Paenibacillus sp. strain NRRL B-50972 BC without any stabilizing agent or mixed with urea, betaine hydrochloride, or potassium phosphate dibasic before and after storage at 54° C. for two weeks. Error bars represent the 95% confidence intervals for the values shown.

FIG. 4 depicts relative fusaricidin levels in liquid formulations of Paenibacillus sp. strain NRRL B-50972 BC without any stabilizing agent or mixed with urea, betaine hydrochloride, or potassium phosphate dibasic before and after storage at 40° C. for eight weeks. Error bars represent the 95% confidence intervals for the values shown.

FIG. 5 depicts relative fusaricidin levels in liquid formulations of Paenibacillus sp. strain NRRL B-50972 BC in a 50:50 dilution with water or propylene glycol before and after storage at 54° C. for two weeks.

FIG. 6 depicts relative fusaricidin levels in liquid formulations of Paenibacillus sp. strain NRRL B-50972 BC in a 50:50 dilution with water or propylene glycol before and after storage at 40° C. for two, four, or eight weeks.

FIG. 7 depicts a photo of a sample of Paenibacillus sp. strain NRRL B-50972 BC without urea (“BC−Urea”) and a sample of Paenibacillus sp. strain NRRL B-50972 BC with urea (“BC+Urea”) after storage at 54° C. for two weeks.

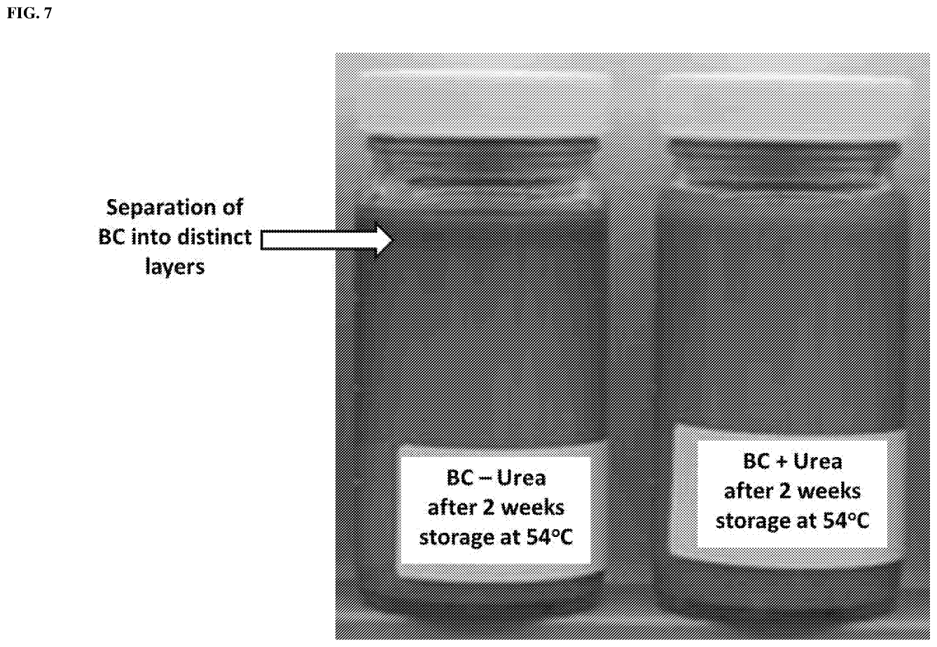

FIG. 8 depicts relative fusaricidin levels in liquid formulations of Paenibacillus sp. strain NRRL B-50972 BC without any stabilizing agent or mixed with urea before and after storage at 23° C. for two, four, eight, twenty-six, fifty-two, and one hundred and four weeks.

FIG. 9 depicts relative fusaricidin levels in liquid formulations of Paenibacillus sp. strain NRRL B-50972 BC without any stabilizing agent or mixed with urea along with Paenibacillus sp. strain NRRL B-67304 BC and Paenibacillus sp. strain NRRL B-67306 BC each mixed with urea before and after storage at 40° C. for four and eight weeks.

FIG. 10 depicts relative fusaricidin levels in liquid formulations of Paenibacillus sp. strain NRRL B-50972 BC without any stabilizing agent or mixed with urea along with Paenibacillus sp. strain NRRL B-67304 BC and Paenibacillus sp. strain NRRL B-67306 BC each mixed with urea before and after storage at 23° C. for two, four, eight, twenty-six, and fifty-two weeks.

FIG. 11 depicts relative fusaricidin levels in liquid formulations of Paenibacillus sp. strains NRRL B-67304, NRRL B-67306, and NRRL B-67615 during storage at 40° C. for eight weeks. The liquid formulations were whole broth without urea (“WB−Urea”) and broth concentrate with urea (“BC+Urea”).

FIG. 12 depicts relative fusaricidin levels in liquid formulations of Paenibacillus sp. strains NRRL B-67304, NRRL B-67306, and NRRL B-67615 during storage at 4° C. or at 23° C. for fifty-two weeks. The liquid formulations were whole broth without urea (“WB−Urea”) and broth concentrate with urea (“BC+Urea”).

### DETAILED DESCRIPTION

It has been found that particular stabilizing agents preserve the antifungal activity of Paenibacillus sp. strains allowing for storage and shipping at elevated temperatures and/or for extended period of times. The Paenibacillus sp. strains include Paenibacillus sp. strain NRRL B-50972, Paenibacillus sp. strain NRRL B-67129, Paenibacillus sp. strain NRRL B-67304, Paenibacillus sp. strain NRRL B-67306, Paenibacillus sp. strain NRRL B-67615, and fungicidal mutants derived therefrom.

Paenibacillus sp. strain NRRL B-50972 and Paenibacillus sp. strain NRRL B-67129 were previously identified as producers of a unique group of fusaricidins and fusaricidin-like compounds with broad spectrum antifungal activity (WO 2016/154297). Paenibacillus sp. strain NRRL B-50972 is related to Paenibacillus sp. strain NRRL B-67304 and Paenibacillus sp. strain NRRL B-67306 as described in U.S. Patent Application No. 62/671,067. Fusaricidin-like compounds include the Paeniserines and Paeniprolixins described in WO 2016/154297. Additional fusaricidin-like compounds include the fusaricidin-type compounds (e.g., compounds 1A, 1B, 2A, 2B, 4A, 4B, 5A, and 5B) described in WO 2016/020371.

As used herein, the verb “comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”.

In certain embodiments, the present invention is directed to a stabilized fungicidal composition comprising: a) a fusaricidin and/or a fusaricidin-like compound; and b) a stabilizing agent selected from the group consisting of an amine, a quaternary ammonium compound, a salt of phosphate or citrate, a short chain polyol having from 2 to 10 carbon atoms, a urea, and combinations thereof.

In other embodiments, the present invention is directed to a stabilized fungicidal composition comprising: a) a fusaricidin and/or a fusaricidin-like compound; and b) a stabilizing agent selected from the group consisting of an amine, a quaternary ammonium compound, a salt of phosphate or citrate, a short chain polyol having from 2 to 10 carbon atoms, a urea, an alkanolamine, an amide, and combinations thereof.

In some embodiments, the present invention is directed to a stabilized fungicidal composition comprising: a) a fusaricidin and/or a fusaricidin-like compound; and b) a stabilizing agent selected from the group consisting of carbonic diamide (CO(NH₂)₂), guanidine hydrochloride, triethanolamine, betaine hydrochloride, potassium phosphate (dibasic), potassium citrate (tribasic), and a combination thereof.

In certain embodiments, the stabilized composition comprises a cell-free preparation of fermentation broth of a Paenibacillus sp. strain. In one aspect, the cell-free preparation comprises a fusaricidin and/or a fusaricidin-like compound.

It should be understood that any numerical range recited herein is intended to include all sub-ranges subsumed therein. For example, a range of 1 to 10 is intended to include all sub-ranges between and including the recited minimum value of 1 and the recited maximum value of 10, that is, having a minimum value equal to or greater than 1 and a maximum value of equal to or less than 10.

The term “alkyl,” as used herein unless otherwise defined, refers to a straight, branched, or cyclic saturated group derived form the removal of a hydrogen atom from an alkane. Representative straight chain alkyl groups include -methyl, -ethyl, -n-propyl, -n-butyl, -n-pentyl, and n-heptyl. Representative branched alkyl groups include -isopropyl, -sec-butyl, -isobutyl, -tert-butyl, -isopentyl, -neopentyl, 1-methylbutyl, 2-methylbutyl, 3-methylbutyl, 1,1-dimethylpropyl and 1,2-dimethylpropyl. Representative cyclic alkyl groups include cyclohexyl, cyclopentyl, and cyclopropyl.

In one embodiment, the stabilizing agent is a “urea”. As used herein, the term “urea” refers to a chemical compound having a functional group made up of a carbonyl group attached to two amine residues wherein each amine residue independently is a primary amine, a secondary amine, or a tertiary amine. The simplest urea is carbonic diamide (CO(NH₂)₂) where both amine residues are primary amines.

In certain aspects, the urea is a substituted urea of formula (I)

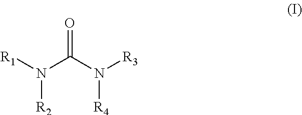

wherein R₁, R₂, and R₃ are independently —H, —OH, C₁-C₆ alkyl, or —R₅OH; R₄ is —R₅OH; and R₅ is C₁-C₆ alkyl.

Illustrative species of the substituted urea are hydroxymethyl urea, hydroxyethyl urea, hydroxypropyl urea; bis (hydroxymethyl) urea; bis (hydroxyethyl) urea; bis (hydroxypropyl) urea; N, N′-di-hydroxymethyl urea; N, N′-di-hydroxyethyl urea; N, N′-di-hydroxypropyl urea; N, N, N′-tri-hydroxyethyl urea; tetra (hydroxymethyl) urea; tetra (hydroxyethyl) urea; tetra (hydroxypropyl) urea; N-methyl-N′-hydroxyethyl urea; N-ethyl-N′-hydroxyethyl urea; N-hydroxypropyl-N′-hydroxyethyl urea and N, N′-dimethyl-N-hydroxyethyl urea. Where the term hydroxypropyl appears, the meaning is generic for either 3-hydroxy-n-propyl, 2-hydroxy-n-propyl, 3-hydroxy-i-propyl or 2-hydroxy-i-propyl radicals.

In some embodiments, the stabilizing agent is carbonic diamide (CO(NH₂)₂) or a substituted urea. In other embodiments, the stabilizing agent is carbonic diamide (CO(NH₂)₂).

In certain aspects, the stabilizing agent in the disclosed fungicidal composition or formulation comprises a urea and a salt of phosphate or citrate. In one aspect, the salt of phosphate or citrate is selected from the group consisting of potassium phosphate monobasic, potassium phosphate dibasic, potassium citrate monobasic, potassium citrate dibasic, and combinations thereof.

As used herein, the term “combined concentration” refers to the sum of the concentrations of the referenced individual components in a composition or formulation. In some embodiments, the urea and the salt of phosphate or citrate are present in the fungicidal composition or formulation at a combined concentration of between about 2% and about 3%. In other embodiments, the urea and the salt of phosphate or citrate are present in the fungicidal composition or formulation at a combined concentration of between about 0.1% and about 3%, between about 0.5% and about 3%, between about 1% and about 3%, or between about 1.5% and about 3%. In yet other embodiments, the urea and the salt of phosphate or citrate are present in the fungicidal composition or formulation at a combined concentration of between about 0.1% and about 2%, between about 0.25% and about 2%, between about 0.5% and about 2%, or between about 1% and about 2%. Non-limiting examples of such mixtures of the urea and the salt of phosphate or citrate are presented in Table 1.

Certain advantages arise from the combination of the urea and the salt of phosphate or citrate in the composition or formulation. Multiple stabilizing agents increases the stability of the fusaricidins and fusaricidin-like compounds, and in case one of the stabilizing agents fails under adverse conditions, another stabilizing agent may still be active and exert a protective effect. In addition, the salts of the polyprotic acids (i.e., the phosphate and citrate salts) provide a pH buffering effect on the composition or formulation that further stabilizes the system.

TABLE 1
Non-limiting examples of combinations of stabilizing agents with an improved effect
of inhibiting the degradation of fusaricidins and fusaricidin-like compounds.

| Stabilizing Agent | Mixture 1 | Mixture 2 | Mixture 3 | Mixture 4 | Mixture 5 | Mixture 6 | Mixture 7 | Mixture 8 |
|---|---|---|---|---|---|---|---|---|
| Urea | 2% to 1% | 2% to 1% | 2% to 1% | 2% to 1% | 2% to 1% | 2% to 1% | 2% to 1% | 2% to 1% |
| Potassium Phosphate | 1% to 2% | — | — | — | 0% to 1% | — | — | — |
| Monobasic (KH₂PO₄) | | | | | | | | |
| Potassium Phosphate | — | 1% to 2% | — | — | — | 0% to 1% | — | — |
| Dibasic (K₂HPO₄) | | | | | | | | |
| Potassium Citrate | — | — | 1% to 2% | — | — | — | 0% to 1% | — |
| Monobasic (C₆H₇KO₇) | | | | | | | | |
| Potassium Citrate | — | — | — | 1% to 2% | — | — | — | 0% to 1% |
| Dibasic (C₆H₆K₂O₇) | | | | | | | | |
| Total Stabilizer | 3% | 3% | 3% | 3% | 2% | 2% | 2% | 2% |
| Concentration | | | | | | | | |

In some aspects, the stabilizing agent is an alkanolamine. As used herein, an “alkanolamine” is a chemical compound containing both hydroxyl (—OH) and amino (i.e., a primary, secondary, or tertiary amino) functional groups on an alkane backbone.

In one aspect, the alkanolamine is an aminoalcohol. In another aspect the aminoalcohol is ethanolamine, diethanolamine, triethanolamine, isopropanolamine, diisopropanolamine (DIPA), triisopropanolamine (TIPA), methanolamine, aminomethyl propanol, heptaminol, dimethylethanolamine, or N-methylethanolamine. In yet another aspect, the aminoalcohol is ethanolamine, diethanolamine, triethanolamine, isopropanolamine, diisopropanolamine (DIPA), triisopropanolamine (TIPA), or methanolamine. In some embodiments, the aminoalochol is ethanolamine, diethanolamine, or triethanolamine. In a certain embodiment, the aminoalochol is triethanolamine.

In yet other embodiments, the stabilizing agent is an amide. In some embodiments, the amide is an organic amide, a sulfonamide, or a phoshoramide.

In one aspect, the organic amide is a carboxamide. In some embodiments, the carboxamide is acetamide or benzamide.

In some embodiments, the disclosed fungicidal composition or formulation further comprises a protease inhibitor. In certain aspects, the protease inhibitor is an aspartic protease inhibitor, cysteine protease inhibitor, metalloprotease inhibitor, serine protease inhibitor, threonine protease inhibitor, or trypsin inhibitor. In one aspect, the trypsin inhibitor is a Kunitz soybean trypsin inhibitor, also known as a Kunitz STI protease inhibitor. In other aspects, the protease inhibitor is a suicide inhibitor, transition state inhibitor, protein protease inhibitor, or chelating agent. In one aspect, the protein protease inhibitor is a serpin.

The microorganisms and particular strains described herein, unless specifically noted otherwise, are all separated from nature and grown under artificial conditions such as in shake flask cultures or through scaled-up manufacturing processes, such as in bioreactors to maximize bioactive metabolite production, for example. Growth under such conditions leads to strain “domestication.” Generally, such a “domesticated” strain differs from its counterparts found in nature in that it is cultured as a homogenous population that is not subject to the selection pressures found in the natural environment but rather to artificial selection pressures.

Microorganisms of the invention, or cultures or isolates thereof, may be described to be in an “isolated” or “biologically pure” form. These terms are intended to mean that the microorganisms have been separated from an environment or one or more constituents, cellular or otherwise, which they may be associated with if found in nature or otherwise. The terms “isolated” or “biologically pure” should not be taken to indicate the extent to which the microorganisms have been purified. However, in one embodiment the isolates or cultures of the microorganisms contain a predominance of the microorganisms of the invention.

Culturing of Paenibacillus sp. strain NRRL B-50972 gives rise to a spontaneous variant strain with a stable colony morphology designated herein as Paenibacillus sp. strain NRRL B-67129. Paenibacillus sp. strain NRRL B-67129 is closely related to Paenibacillus sp. strain NRRL B-50972 except that Paenibacillus sp. strain NRRL B-67129 expresses Spo0A with a single amino acid substitution compared to the Spo0A expressed by Paenibacillus sp. strain NRRL B-50972.

In one embodiment, a mutant strain of the Paenibacillus sp. strain NRRL B-50972, Paenibacillus sp. strain NRRL B-67129, Paenibacillus sp. strain NRRL B-67304, Paenibacillus sp. strain NRRL B-67306, or Paenibacillus sp. strain NRRL B-67615 is provided. The term “mutant” refers to a genetic variant derived from a Paenibacillus sp. strain. In one embodiment, the mutant has one or more or all the identifying (functional) characteristics of the Paenibacillus sp. strain. In a particular instance, the mutant or a fermentation product thereof controls (as an identifying functional characteristic) fungi and/or bacteria at least as well as the parent Paenibacillus sp. strain. Such mutants may be genetic variants having a genomic sequence that has greater than about 85%, greater than about 90%, greater than about 95%, greater than about 98%, or greater than about 99% sequence identity to the parent Paenibacillus sp. strain. Mutants may be obtained by treating cells of a Paenibacillus sp. strain with chemicals or irradiation or by selecting spontaneous mutants from a population of a Paenibacillus sp. strain cells (such as phage resistant or antibiotic resistant mutants), by genome shuffling, as described below, or by other means well known to those practiced in the art.

Genome shuffling among Paenibacillus strains can be facilitated through the use of a process called protoplast fusion. The process begins with the formation of protoplasts from vegetative bacillary cells. The removal of peptidoglycan cell wall, typically using lysozyme and an osmotic stabilizer, results in the formation of a protoplast. This process is visible under a light microscope with the appearance of spherical cells. Addition of PEG, polyethylene glycol, then induces fusion among protoplasts, allowing genetic contents of two or more cells to come in contact facilitating recombination and genome shuffling. Fused cells then repartition and are recovered on a solid growth medium. During recovery, protoplasts rebuild peptidoglycan cell walls, transitioning back to bacillary shape. See Schaeffer, et. al., (1976) PNAS USA, vol. 73, 6:2151-2155).

The Paenibacillus sp. strain NRRL B-50972, Paenibacillus sp. strain NRRL B-67129, Paenibacillus sp. strain NRRL B-67304, Paenibacillus sp. strain NRRL B-67306, Paenibacillus sp. strain NRRL B-67615, and mutants thereof have activity against a broad range of plant pathogens. In one aspect, the strains has activity against fungi, such as cucumber anthracnose, cucumber powdery mildew, wheat leaf rust, barley powdery mildew, Alternaria, and Botrytis; Oomycetes, such as tomato late blight, cucumber downy mildew and brassica downy mildew; and/or bacteria, such as Pseudomonas, Xanthomonas, and Erwinia.

The present invention also encompasses methods of treating a plant to control plant diseases by administering to a plant or a plant part, such as a leaf, stem, flowers, fruit, root, or seed or by applying to a locus on which plant or plant parts grow, such as soil, aqueous formulations or fungicidal compositions comprising a fusaricidin-producing Paenibacillus sp. strain and a stabilizing agent selected from the group consisting of urea, guanidine hydrochloride, triethanolamine, betaine hydrochloride, potassium phosphate (dibasic), potassium citrate (tribasic), and a combination thereof.

In a method according to the invention the composition or formulation can be applied to any plant or any part of any plant grown in any type of media used to grow plants (e.g., soil, vermiculite, shredded cardboard, and water) or applied to plants or the parts of plants grown aerially, such as orchids or staghorn ferns. The composition or formulation may for instance be applied by spraying, atomizing, vaporizing, scattering, dusting, watering, squirting, sprinkling, pouring or fumigating. As already indicated above, application may be carried out at any desired location where the plant of interest is positioned, such as agricultural, horticultural, forest, plantation, orchard, nursery, organically grown crops, turfgrass and urban environments.

Compositions of the present invention can be obtained by culturing a fusaricidin-producing Paenibacillus sp. strain according to methods well known in the art, including by using the media and other methods described in the examples below. Conventional large-scale microbial culture processes include submerged fermentation, solid state fermentation, or liquid surface culture. Towards the end of fermentation, as nutrients are depleted, cells begin the transition from growth phase to sporulation phase, such that the final product of fermentation is largely spores, metabolites and residual fermentation medium. Sporulation is part of the natural life cycle of Paenibacillus and is generally initiated by the cell in response to nutrient limitation. Fermentation is configured to obtain high levels of colony forming units of and to promote sporulation. The bacterial cells, spores and metabolites in culture media resulting from fermentation may be used directly or concentrated by conventional industrial methods, such as centrifugation, tangential-flow filtration, depth filtration, and evaporation.

Compositions of the present invention include fermentation products. In some embodiments, the concentrated fermentation broth is washed, for example, via a diafiltration process, to remove residual fermentation broth and metabolites. The term “broth concentrate,” as used herein, refers to whole broth (fermentation broth) that has been concentrated by conventional industrial methods, as described above, but remains in liquid form. The term “fermentation solid,” as used herein, refers to the solid material that remains after the fermentation broth is dried. The term “fermentation product,” as used herein, refers to whole broth, broth concentrate and/or fermentation solids. Compositions of the present invention include fermentation products.

Cell-free preparations of fermentation broth of the strains of the present invention can be obtained by any means known in the art, such as extraction, centrifugation and/or filtration of fermentation broth. Those of skill in the art will appreciate that so-called cell-free preparations may not be devoid of cells but rather are largely cell-free or essentially cell-free, depending on the technique used (e.g., speed of centrifugation) to remove the cells. The resulting cell-free preparation may be formulated with components that aid in its application to plants or to plant growth media. Concentration methods and drying techniques described above for fermentation broth are also applicable to cell-free preparations.

In one embodiment, the fermentation product comprises at least about 1×10⁴ colony forming units (CFU) of the microorganism (e.g., Paenibacillus sp. strain NRRL B-50972 or a fungicidal mutant strain thereof)/mL broth. In another embodiment, the fermentation product comprises at least about 1×10⁵ colony forming units (CFU) of the microorganism (e.g., Paenibacillus sp. strain NRRL B-50972 or a fungicidal mutant strain thereof)/mL broth. In another embodiment, the fermentation product comprises at least about 1×10⁶ CFU of the microorganism (e.g., Paenibacillus sp. strain NRRL B-50972 or a fungicidal mutant strain thereof)/mL broth. In yet another embodiment, the fermentation product comprises at least about 1×10⁷ CFU of the microorganism (e.g., Paenibacillus sp. strain NRRL B-50972 or a fungicidal mutant strain thereof)/mL broth. In another embodiment, the fermentation product comprises at least about 1×10⁸ CFU of the microorganism (e.g., Paenibacillus sp. strain NRRL B-50972 or a fungicidal mutant strain thereof)/mL broth. In another embodiment, the fermentation product comprises at least about 1×10⁹ CFU of the microorganism (e.g., Paenibacillus sp. strain NRRL B-50972 or a fungicidal mutant strain thereof)/mL broth. In another embodiment, the fermentation product comprises at least about 1×10¹⁰ CFU of the microorganism (e.g., Paenibacillus sp. strain NRRL B-50972 or a fungicidal mutant strain thereof)/mL broth. In another embodiment, the fermentation product comprises at least about 1×10¹¹ CFU of the microorganism (e.g., Paenibacillus sp. strain NRRL B-50972 or a fungicidal mutant strain thereof)/mL broth.

The inventive compositions can be used as such or, depending on their particular physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, gas (under pressure), gas generating product, foams, pastes, pesticide coated seed, suspension concentrates, oil dispersion, suspoemulsion concentrates, soluble concentrates, suspensions, wettable powders, soluble powders, dusts and granules, water-soluble and water-dispersible granules or tablets, water-soluble and water-dispersible powders for the treatment of seed, natural products and synthetic substances impregnated with active ingredient, and also microencapsulations in polymeric substances and in coating materials for seed, and also ULV cold-fogging and warm-fogging formulations.

In some embodiments, the inventive compositions are liquid formulations. Non-limiting examples of liquid formulations include suspension concentrations and oil dispersions.

Compositions of the present invention may include formulation inerts added to compositions comprising cells, cell-free preparations or metabolites to improve efficacy, stability, and usability and/or to facilitate processing, packaging and end-use application. Such formulation inerts and ingredients may include carriers, stabilization agents, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the carrier is a binder or adhesive that facilitates adherence of the composition to a plant part, such as a seed or root. See, for example, Taylor, A. G., et al., “Concepts and Technologies of Selected Seed Treatments”, Annu. Rev. Phytopathol. 28: 321-339 (1990). The stabilization agents may include anti-caking agents, anti-oxidation agents, desiccants, protectants or preservatives. The nutrients may include carbon, nitrogen, and phosphors sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, antifreeze agents or colorants. In some embodiments, the composition comprising cells, cell-free preparation or metabolites produced by fermentation can be used directly with or without water as the diluent without any other formulation preparation. In some embodiments, the formulation inerts are added after concentrating fermentation broth and during and/or after drying.

All plants and plant parts can be treated in accordance with the invention. In the present context, plants are understood as meaning all plants and plant populations, such as desired and undesired wild plants or crop plants (including naturally occurring crop plants). Crop plants can be plants which can be obtained by traditional breeding and optimization methods or by biotechnological and recombinant methods, or combinations of these methods, including the transgenic plants and including the plant varieties capable or not of being protected by Plant Breeders' Rights. Plant parts are understood as meaning all aerial and subterranean parts and organs of the plants, such as shoot, leaf, flower and root, examples which may be mentioned being leaves, needles, stalks, stems, flowers, fruiting bodies, fruits and seeds, and also roots, tubers and rhizomes. The plant parts also include crop material and vegetative and generative propagation material, for example cuttings, tubers, rhizomes, slips and seeds.

As has already been mentioned above, all plants and their parts may be treated in accordance with the invention. In a preferred embodiment, plant species and plant varieties, and their parts, which grow wild or which are obtained by traditional biological breeding methods such as hybridization or protoplast fusion are treated. In a further preferred embodiment, transgenic plants and plant varieties which have been obtained by recombinant methods, if appropriate in combination with traditional methods (genetically modified organisms), and their parts are treated. The term “parts” or “parts of plants” or “plant parts” has been explained hereinabove. Plants of the plant varieties which are in each case commercially available or in use are especially preferably treated in accordance with the invention. Plant varieties are understood as meaning plants with novel traits which have been bred both by traditional breeding, by mutagenesis or by recombinant DNA techniques. They may take the form of varieties, races, biotypes and genotypes.

The treatment of the plants and plant parts with the compositions according to the invention is carried out directly or by acting on the environment, habitat or storage space using customary treatment methods, for example by dipping, spraying, atomizing, misting, evaporating, dusting, fogging, scattering, foaming, painting on, spreading, injecting, drenching, trickle irrigation and, in the case of propagation material, in particular in the case of seed, furthermore by the dry seed treatment method, the wet seed treatment method, the slurry treatment method, by encrusting, by coating with one or more coats and the like. It is furthermore possible to apply the active substances by the ultra-low volume method or to inject the active substance preparation or the active substance itself into the soil.

A preferred direct treatment of the plants is the leaf application treatment, i.e., compositions according to the invention are applied to the foliage, it being possible for the treatment frequency and the application rate to be matched to the infection pressure of the pathogen in question.

In the case of systemically active compounds, the compositions according to the invention reach the plants via the root system. In this case, the treatment of the plants is effected by allowing the compositions according to the invention to act on the environment of the plant. This can be done for example by drenching, incorporating in the soil or into the nutrient solution, i.e., the location of the plant (for example the soil or hydroponic systems) is impregnated with a liquid form of the compositions according to the invention, or by soil application, i.e., the compositions according to the invention are incorporated into the location of the plants in solid form (for example in the form of granules). In the case of paddy rice cultures, this may also be done by metering the compositions according to the invention into a flooded paddy field in a solid use form (for example in the form of granules).

Preferred plants are those from the group of the useful plants, ornamentals, turfs, generally used trees which are employed as ornamentals in the public and domestic sectors, and forestry trees. Forestry trees comprise trees for the production of timber, cellulose, paper and products made from parts of the trees.

The term “useful plants” as used in the present context refers to crop plants which are employed as plants for obtaining foodstuffs, feedstuffs, fuels or for industrial purposes.

The useful plants which can be treated and/or improved with the compositions and methods of the present invention include for example the following types of plants: turf, vines, cereals, for example wheat, barley, rye, oats, rice, maize and millet/sorghum; beet, for example sugar beet and fodder beet; fruits, for example pome fruit, stone fruit and soft fruit, for example apples, pears, plums, peaches, almonds, cherries and berries, for example strawberries, raspberries, blackberries; legumes, for example beans, lentils, peas and soybeans; oil crops, for example oilseed rape, mustard, poppies, olives, sunflowers, coconuts, castor oil plants, cacao and peanuts; cucurbits, for example pumpkin/squash, cucumbers and melons; fibre plants, for example cotton, flax, hemp and jute; citrus fruit, for example oranges, lemons, grapefruit and tangerines; vegetables, for example spinach, lettuce, asparagus, cabbage species, carrots, onions, tomatoes, potatoes and bell peppers; Lauraceae, for example avocado, Cinnamomum, camphor, or else plants such as tobacco, nuts, coffee, aubergine, sugar cane, tea, pepper, grapevines, hops, bananas, latex plants and ornamentals, for example flowers, shrubs, deciduous trees and coniferous trees. This enumeration is no limitation.

The following plants are considered to be particularly suitable target crops for applying compositions and methods of the present invention: cotton, aubergine, turf, pome fruit, stone fruit, soft fruit, maize, wheat, barley, cucumber, tobacco, vines, rice, cereals, pear, beans, soybeans, oilseed rape, tomato, bell pepper, melons, cabbage, potato and apple.

The present invention can also be applied to any turf grasses, including cool-season turf grasses and warm-season turf grasses. Examples of cold-season turf grasses are bluegrasses (Poa ssp.), such as Kentucky bluegrass (Poa pratensis L.), rough bluegrass (Poa trivialis L.), Canada bluegrass (Poa compressa L.), annual bluegrass (Poa annua L.), upland bluegrass (Poa glaucantha Gaudin), wood bluegrass (Poa nemoralis L.) and bulbous bluegrass (Poa bulbosa L.); bentgrasses (Agrostis ssp.) such as creeping bentgrass (Agrostis palustris Huds.), colonial bentgrass (Agrostis tenuis Sibth.), velvet bentgrass (Agrostis canina L.), South German mixed bentgrass (Agrostis ssp. including Agrostis tenuis Sibth., Agrostis canina L., and Agrostis palustris Huds.), and redtop (Agrostis alba L.);

fescues (Festuca ssp.), such as red fescue (Festuca rubra L. ssp. rubra), creeping fescue (Festuca rubra L.), chewings fescue (Festuca rubra commutata Gaud.), sheep fescue (Festuca ovina L.), hard fescue (Festuca longifolia Thuill.), hair fescue (Festucu capillata Lam.), tall fescue (Festuca arundinacea Schreb.) and meadow fescue (Festuca elanor L.);

ryegrasses (Lolium ssp.), such as annual ryegrass (Lolium multiflorum Lam.), perennial ryegrass (Lolium perenne L.) and Italian ryegrass (Lolium multiflorum Lam.);

and wheatgrasses (Agropyron ssp.), such as fairway wheatgrass (Agropyron cristatum (L.) Gaertn.), crested wheatgrass (Agropyron desertorum (Fisch.) Schult.) and western wheatgrass (Agropyron smithii Rydb.)

Examples of further cool-season turf grasses are beachgrass (Ammophila breviligulata Fern.), smooth bromegrass (Bromus inermis Leyss.), cattails such as timothy (Phleum pratense L.), sand cattail (Phleum subulatum L.), orchardgrass (Dactylis glomerata L.), weeping alkaligrass (Puccinellia distans (L.) Parl.) and crested dog's-tail (Cynosurus cristatus L.)

Examples of warm-season turf grasses are Bermuda grass (Cynodon ssp. L. C. Rich), zoysia grass (Zoysia ssp. Willd.), St. Augustine grass (Stenotaphrum secundatum Walt Kuntze), centipede grass (Eremochloa ophiuroides Munro Hack.), carpetgrass (Axonopus affnis Chase), Bahia grass (Paspalum notatum Flugge), Kikuyu grass (Pennisetum clandestinum Hochst. ex Chiov.), buffalo grass (Buchloe dactyloids (Nutt.) Engelm.), blue grama (Bouteloua gracilis (H.B.K.) Lag. ex Griffiths), seashore paspalum (Paspalum vaginatum Swartz) and sideoats grama (Bouteloua curtipendula (Michx. Torr.) Cool-season turf grasses are generally preferred for the use according to the invention. Especially preferred are bluegrass, benchgrass and redtop, fescues and ryegrasses. Bentgrass is especially preferred.

The inventive compositions have potent microbicidal activity and can be used for control of unwanted microorganisms, such as fungi and bacteria, in crop protection and in the protection of materials.

The invention also relates to a method for controlling unwanted microorganisms, characterized in that the inventive compositions are applied to the phytopathogenic fungi, phytopathogenic bacteria and/or their habitat.

Fungicides can be used in crop protection for control of phytopathogenic fungi. They are characterized by an outstanding efficacy against a broad spectrum of phytopathogenic fungi, including soilborne pathogens, which are in particular members of the classes Plasmodiophoromycetes, Peronosporomycetes (Syn. Oomycetes), Chytridiomycetes, Zygomycetes, Ascomycetes, Basidiomycetes and Deuteromycetes (Syn. Fungi imperfecti). Some fungicides are systemically active and can be used in plant protection as foliar, seed dressing or soil fungicide. Furthermore, they are suitable for combating fungi, which inter alia infest wood or roots of plant.

Bactericides can be used in crop protection for control of Pseudomonadaceae, Rhizobiaceae, Enterobacteriaceae, Corynebacteriaceae and Streptomycetaceae.

Non-limiting examples of pathogens of fungal diseases which can be treated in accordance with the invention include:

diseases caused by powdery mildew pathogens, for example Blumeria species, for example Blumeria graminis; Podosphaera species, for example Podosphaera leucotricha; Sphaerotheca species, for example Sphaerotheca fuliginea; Uncinula species, for example Uncinula necator;

diseases caused by rust disease pathogens, for example Gymnosporangium species, for example Gymnosporangium sabinae; Hemileia species, for example Hemileia vastatrix; Phakopsora species, for example Phakopsora pachyrhizi and Phakopsora meibomiae; Puccinia species, for example Puccinia recondite, P. triticina, P. graminis or P. striiformis; Uromyces species, for example Uromyces appendiculatus;

diseases caused by pathogens from the group of the Oomycetes, for example Albugo species, for example Albugo candida; Bremia species, for example Bremia lactucae; Peronospora species, for example Peronospora pisi or P. brassicae; Phytophthora species, for example Phytophthora infestans; Plasmopara species, for example Plasmopara viticola; Pseudoperonospora species, for example Pseudoperonospora humuli or Pseudoperonospora cubensis; Pythium species, for example Pythium ultimum;

leaf blotch diseases and leaf wilt diseases caused, for example, by Alternaria species, for example Alternaria solani; Cercospora species, for example Cercospora beticola; Cladiosporium species, for example Cladiosporium cucumerinum; Cochliobolus species, for example Cochliobolus sativus (conidia form: Drechslera, Syn: Helminthosporium), Cochliobolus miyabeanus; Colletotrichum species, for example Colletotrichum lindemuthanium; Cycloconium species, for example Cycloconium oleaginum; Diaporthe species, for example Diaporthe citri; Elsinoe species, for example Elsinoe fawcettii; Gloeosporium species, for example Gloeosporium laeticolor; Glomerella species, for example Glomerella cingulata; Guignardia species, for example Guignardia bidwelli; Leptosphaeria species, for example Leptosphaeria maculans, Leptosphaeria nodorum; Magnaporthe species, for example Magnaporthe grisea; Marssonia species, for example Marssonia coronaria; Microdochium species, for example Microdochium nivale; Mycosphaerella species, for example Mycosphaerella graminicola, M. arachidicola and M. fijiensis; Phaeosphaeria species, for example Phaeosphaeria nodorum; Pyrenophora species, for example Pyrenophora teres, Pyrenophora tritici repentis; Ramularia species, for example Ramularia collo-cygni, Ramularia areola; Rhynchosporium species, for example Rhynchosporium secalis; Septoria species, for example Septoria apii, Septoria lycopersii; Typhula species, for example Typhula incarnata; Venturia species, for example Venturia inaequalis;

root and stem diseases caused, for example, by Corticium species, for example Corticium graminearum; Fusarium species, for example Fusarium oxysporum; Gaeumannomyces species, for example Gaeumannomyces graminis; Rhizoctonia species, such as, for example Rhizoctonia solani; Sarocladium diseases caused for example by Sarocladium oryzae; Sclerotium diseases caused for example by Sclerotium oryzae; Tapesia species, for example Tapesia acuformis; Thielaviopsis species, for example Thielaviopsis basicola;

ear and panicle diseases (including corn cobs) caused, for example, by Alternaria species, for example Alternaria ssp.; Aspergillus species, for example Aspergillus flavus; Cladosporium species, for example Cladosporium cladosporioides; Claviceps species, for example Claviceps purpurea; Fusarium species, for example Fusarium culmorum; Gibberella species, for example Gibberella zeae; Monographella species, for example Monographella nivalis; Septoria species, for example Septoria nodorum;

diseases caused by smut fungi, for example Sphacelotheca species, for example Sphacelotheca reiliana; Tilletia species, for example Tilletia caries, T. controversa; Urocystis species, for example Urocystis occulta; Ustilago species, for example Ustilago nuda, U. nuda tritici;

fruit rot caused, for example, by Aspergillus species, for example Aspergillus flavus; Botrytis species, for example Botrytis cinerea; Penicillium species, for example Penicillium expansum and P. purpurogenum; Sclerotinia species, for example Sclerotinia sclerotiorum; Verticilium species, for example Verticilium alboatrum;

seed and soilborne decay, mould, wilt, rot and damping-off diseases caused, for example, by Alternaria species, caused for example by Alternaria brassicicola; Aphanomyces species, caused for example by Aphanomyces euteiches; Ascochyta species, caused for example by Ascochyta lentis; Aspergillus species, caused for example by Aspergillus flavus; Cladosporium species, caused for example by Cladosporium herbarum; Cochliobolus species, caused for example by Cochliobolus sativus; (Conidiaform: Drechslera, Bipolaris Syn: Helminthosporium); Colletotrichum species, caused for example by Colletotrichum coccodes; Fusarium species, caused for example by Fusarium culmorum; Gibberella species, caused for example by Gibberella zeae; Macrophomina species, caused for example by Macrophomina phaseolina; Monographella species, caused for example by Monographella nivalis; Penicillium species, caused for example by Penicillium expansum; Phoma species, caused for example by Phoma lingam; Phomopsis species, caused for example by Phomopsis sojae; Phytophthora species, caused for example by Phytophthora cactorum; Pyrenophora species, caused for example by Pyrenophora graminea; Pyricularia species, caused for example by Pyricularia oryzae; Pythium species, caused for example by Pythium ultimum; Rhizoctonia species, caused for example by Rhizoctonia solani; Rhizopus species, caused for example by Rhizopus oryzae; Sclerotium species, caused for example by Sclerotium rolfsii; Septoria species, caused for example by Septoria nodorum; Typhula species, caused for example by Typhula incarnata; Verticillium species, caused for example by Verticillium dahliae;

cancers, galls and witches' broom caused, for example, by Nectria species, for example Nectria galligena;

wilt diseases caused, for example, by Monilinia species, for example Monilinia laxa;

leaf blister or leaf curl diseases caused, for example, by Exobasidium species, for example Exobasidium vexans;

Taphrina species, for example Taphrina deformans;

decline diseases of wooden plants caused, for example, by Esca disease, caused for example by Phaemoniella clamydospora, Phaeoacremonium aleophilum and Fomitiporia mediterranea; Eutypa dyeback, caused for example by Eutypa lata; Ganoderma diseases caused for example by Ganoderma boninense; Rigidoporus diseases caused for example by Rigidoporus lignosus;

diseases of flowers and seeds caused, for example, by Botrytis species, for example Botrytis cinerea;

diseases of plant tubers caused, for example, by Rhizoctonia species, for example Rhizoctonia solani; Helminthosporium species, for example Helminthosporium solani;

Club root caused, for example, by Plasmodiophora species, for example Plamodiophora brassicae;

diseases caused by bacterial pathogens, for example Xanthomonas species, for example Xanthomonas campestris pv. oryzae; Pseudomonas species, for example Pseudomonas syringae pv. lachrymans; Erwinia species, for example Erwinia amylovora.

The following diseases of soya beans can be controlled with preference:

Fungal diseases on leaves, stems, pods and seeds caused, for example, by Alternaria leaf spot (Alternaria spec. atrans tenuissima), Anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi, Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping-off (Rhizoctonia solani), sclerotinia stem decay (Sclerotinia sclerotiorum), sclerotinia southern blight (Sclerotinia rolfsii), thielaviopsis root rot (Thielaviopsis basicola).

The inventive fungicidal compositions can be used for curative or protective/preventive control of phytopathogenic fungi. The invention therefore also relates to curative and protective methods for controlling phytopathogenic fungi by the use of the inventive compositions, which are applied to the seed, the plant or plant parts, the fruit or the soil in which the plants grow.

The fact that the compositions are well tolerated by plants at the concentrations required for controlling plant diseases allows the treatment of above-ground parts of plants, of propagation stock and seeds, and of the soil.

According to the invention all plants and plant parts can be treated including cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random and directed mutagenesis, molecular or genetic markers or by bioengineering and genetic engineering methods.

In certain aspects, the compositions of the present invention are applied at about 1×10⁸ to about 1×10¹⁴ colony forming units (CFU) of fungicidal Paenibacillus sp. strain NRRL B-50972 or fungicidal mutant strain thereof per hectare. In other aspects, the compositions of the present invention are applied at about 1×10⁹ to about 1×10¹³ colony forming units (CFU) of fungicidal Paenibacillus sp. strain NRRL B-50972 or fungicidal mutant strain thereof per hectare. In yet other aspects, the compositions of the present invention are applied at about 1×10¹⁰ to about 1×10¹² colony forming units (CFU) of fungicidal Paenibacillus sp. strain NRRL B-50972 or fungicidal mutant strain thereof per hectare.

In some embodiments, the compositions of the present invention are applied at about 0.1 kg to about 10 kg fermentation solids per hectare. In other embodiments, the compositions of the present invention are applied at about 0.25 kg to about 7.5 kg fermentation solids per hectare. In yet other embodiments, the compositions of the present invention are applied at about 0.5 kg to about 5 kg fermentation solids per hectare. The compositions of the present invention may also be applied at about 1 kg or about 2 kg fermentation solids per hectare.

The inventive compositions, when they are well tolerated by plants, have favorable homeotherm toxicity and are well tolerated by the environment, are suitable for protecting plants and plant organs, for enhancing harvest yields, for improving the quality of the harvested material. They can preferably be used as crop protection compositions. They are active against normally sensitive and resistant species and against all or some stages of development.

Plants which can be treated in accordance with the invention include the following main crop plants: maize, soya bean, alfalfa, cotton, sunflower, Brassica oil seeds such as Brassica napus (e.g., canola, rapeseed), Brassica rapa, B. juncea (e.g., (field) mustard) and Brassica carinata, Arecaceae sp. (e.g., oilpalm, coconut), rice, wheat, sugar beet, sugar cane, oats, rye, barley, millet and sorghum, triticale, flax, nuts, grapes and vine and various fruit and vegetables from various botanic taxa, e.g., Rosaceae sp. (e.g., pome fruits such as apples and pears, but also stone fruits such as apricots, cherries, almonds, plums and peaches, and berry fruits such as strawberries, raspberries, red and black currant and gooseberry), Ribesioidae sp., Juglandaceae sp., Betulaceae sp., Anacardiaceae sp., Fagaceae sp., Moraceae sp., Oleaceae sp. (e.g., olive tree), Actinidaceae sp., Lauraceae sp. (e.g., avocado, cinnamon, camphor), Musaceae sp. (e.g., banana trees and plantations), Rubiaceae sp. (e.g., coffee), Theaceae sp. (e.g., tea), Sterculiceae sp., Rutaceae sp. (e.g., lemons, oranges, mandarins and grapefruit); Solanaceae sp. (e.g., tomatoes, potatoes, peppers, capsicum, aubergines, tobacco), Liliaceae sp., Compositae sp. (e.g., lettuce, artichokes and chicory—including root chicory, endive or common chicory), Umbelliferae sp. (e.g., carrots, parsley, celery and celeriac), Cucurbitaceae sp. (e.g., cucumbers—including gherkins, pumpkins, watermelons, calabashes and melons), Alliaceae sp. (e.g., leeks and onions), Cruciferae sp. (e.g., white cabbage, red cabbage, broccoli, cauliflower, Brussels sprouts, pak choi, kohlrabi, radishes, horseradish, cress and chinese cabbage), Leguminosae sp. (e.g., peanuts, peas, lentils and beans—e.g., common beans and broad beans), Chenopodiaceae sp. (e.g., Swiss chard, fodder beet, spinach, beetroot), Linaceae sp. (e.g., hemp), Cannabeacea sp. (e.g., cannabis), Malvaceae sp. (e.g., okra, cocoa), Papaveraceae (e.g., poppy), Asparagaceae (e.g., asparagus); useful plants and ornamental plants in the garden and woods including turf, lawn, grass and Stevia rebaudiana; and in each case genetically modified types of these plants.

In certain aspects, the fermentation product further comprises a formulation ingredient. The formulation ingredient may be a wetting agent, extender, solvent, spontaneity promoter, emulsifier, dispersant, frost protectant, thickener, and/or an adjuvant. In one embodiment, the formulation ingredient is a wetting agent. In other aspects, the fermentation product is a freeze-dried powder or a spray-dried powder.

Compositions of the present invention may include formulation ingredients added to compositions of the present invention to improve recovery, efficacy, or physical properties and/or to aid in processing, packaging and administration. Such formulation ingredients may be added individually or in combination.

The formulation ingredients may be added to compositions comprising cells, cell-free preparations, isolated compounds, and/or metabolites to improve efficacy, stability, and physical properties, usability and/or to facilitate processing, packaging and end-use application. Such formulation ingredients may include agriculturally acceptable carriers, inerts, stabilization agents, preservatives, nutrients, or physical property modifying agents, which may be added individually or in combination. In some embodiments, the carriers may include liquid materials such as water, oil, and other organic or inorganic solvents and solid materials such as minerals, polymers, or polymer complexes derived biologically or by chemical synthesis. In some embodiments, the formulation ingredient is a binder, adjuvant, or adhesive that facilitates adherence of the composition to a plant part, such as leaves, seeds, or roots. See, for example, Taylor, A. G., et al., “Concepts and Technologies of Selected Seed Treatments,” Annu. Rev. Phytopathol., 28: 321-339 (1990). The stabilization agents may include anti-caking agents, anti-oxidation agents, anti-settling agents, antifoaming agents, desiccants, protectants or preservatives. The nutrients may include carbon, nitrogen, and phosphorus sources such as sugars, polysaccharides, oil, proteins, amino acids, fatty acids and phosphates. The physical property modifiers may include bulking agents, wetting agents, thickeners, pH modifiers, rheology modifiers, dispersants, adjuvants, surfactants, film-formers, hydrotropes, builders, antifreeze agents or colorants. In some embodiments, the composition comprising cells, cell-free preparation and/or metabolites produced by fermentation can be used directly with or without water as the diluent without any other formulation preparation. In a particular embodiment, a wetting agent, or a dispersant, is added to a fermentation solid, such as a freeze-dried or spray-dried powder. A wetting agent increases the spreading and penetrating properties, or a dispersant increases the dispersibility and solubility of the active ingredient (once diluted) when it is applied to surfaces. Exemplary wetting agents are known to those of skill in the art and include sulfosuccinates and derivatives, such as MULTIWET™ MO-70R (Croda Inc., Edison, N.J.); siloxanes such as BREAK-THRU® (Evonik, Germany); nonionic compounds, such as ATLOX™ 4894 (Croda Inc., Edison, N.J.); alkyl polyglucosides, such as TERWET® 3001 (Huntsman International LLC, The Woodlands, Tex.); C12-C14 alcohol ethoxylate, such as TERGITOL® 15-S-15 (The Dow Chemical Company, Midland, Mich.); phosphate esters, such as RHODAFAC® BG-510 (Rhodia, Inc.); and alkyl ether carboxylates, such as EMULSOGEN™ LS (Clariant Corporation, North Carolina).

The present invention further provides formulations, and application forms prepared from them, as crop protection agents and/or pesticidal agents, such as drench, drip and spray liquors, comprising at least one of the active compounds of the invention. The application forms may comprise further crop protection agents and/or pesticidal agents, and/or activity-enhancing adjuvants such as penetrants, examples being vegetable oils such as, for example, rapeseed oil, sunflower oil, mineral oils such as, for example, liquid paraffins, alkyl esters of vegetable fatty acids, such as rapeseed oil or soybean oil methyl esters, or alkanol alkoxylates, and/or spreaders such as, for example, alkylsiloxanes and/or salts, examples being organic or inorganic ammonium or phosphonium salts, examples being ammonium sulphate or diammonium hydrogen phosphate, and/or retention promoters such as dioctyl sulphosuccinate or hydroxypropylguar polymers and/or humectants such as glycerol and/or fertilizers such as ammonium, potassium or phosphorous fertilizers, for example.

The formulations or application forms in question preferably comprise auxiliaries, such as extenders, solvents, spontaneity promoters, carriers, emulsifiers, dispersants, frost protectants, biocides, thickeners and/or other auxiliaries, such as adjuvants, for example. An adjuvant in this context is a component which enhances the biological effect of the formulation, without the component itself having a biological effect. Examples of adjuvants are agents which promote the retention, spreading, attachment to the leaf surface, or penetration.

These formulations are produced in a known manner, for example by mixing the active compounds with auxiliaries such as, for example, extenders, solvents and/or solid carriers and/or further auxiliaries, such as, for example, surfactants.

Suitable for use as auxiliaries are substances which are suitable for imparting to the formulation of the active compound or the application forms prepared from these formulations (such as, e.g., usable crop protection agents, such as spray liquors or seed dressings) particular properties such as certain physical, technical and/or biological properties.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethylformamide and dimethyl sulphoxide, and also water.

In principle it is possible to use all suitable solvents. Suitable solvents are, for example, aromatic hydrocarbons, such as xylene, toluene or alkylnaphthalenes, for example, chlorinated aromatic or aliphatic hydrocarbons, such as chlorobenzene, chloroethylene or methylene chloride, for example, aliphatic hydrocarbons, such as cyclohexane, for example, paraffins, petroleum fractions, mineral and vegetable oils, alcohols, such as methanol, ethanol, isopropanol, butanol or glycol, for example, and also their ethers and esters, ketones such as acetone, methyl ethyl ketone, methyl isobutyl ketone or cyclohexanone, for example, strongly polar solvents, such as dimethyl sulphoxide, and water.

All suitable carriers may in principle be used. Suitable carriers are in particular: for example, ammonium salts and ground natural minerals such as kaolins, clays, talc, chalk, quartz, attapulgite, montmorillonite or diatomaceous earth, and ground synthetic minerals, such as finely divided silica, alumina and natural or synthetic silicates, resins, waxes and/or solid fertilizers. Mixtures of such carriers may likewise be used. Carriers suitable for granules include the following: for example, crushed and fractionated natural minerals such as calcite, marble, pumice, sepiolite, dolomite, and also synthetic granules of inorganic and organic meals, and also granules of organic material such as sawdust, paper, coconut shells, maize cobs and tobacco stalks.

Liquefied gaseous extenders or solvents may also be used. Particularly suitable are those extenders or carriers which at standard temperature and under standard pressure are gaseous, examples being aerosol propellants, such as halogenated hydrocarbons, and also butane, propane, nitrogen and carbon dioxide.

Examples of emulsifiers and/or foam-formers, dispersants or wetting agents having ionic or nonionic properties, or mixtures of these surface-active substances, are salts of polyacrylic acid, salts of lignosulphonic acid, salts of phenolsulphonic acid or naphthalenesulphonic acid, polycondensates of ethylene oxide with fatty alcohols or with fatty acids or with fatty amines, with substituted phenols (preferably alkylphenols or arylphenols), salts of sulphosuccinic esters, taurine derivatives (preferably alkyltaurates), phosphoric esters of polyethoxylated alcohols or phenols, fatty acid esters of polyols, and derivatives of the compounds containing sulphates, sulphonates and phosphates, examples being alkylaryl polyglycol ethers, alkylsulphonates, alkyl sulphates, arylsulphonates, protein hydrolysates, lignin-sulphite waste liquors and methylcellulose. The presence of a surface-active substance is advantageous if one of the active compounds and/or one of the inert carriers is not soluble in water and if application takes place in water.

Further auxiliaries that may be present in the formulations and in the application forms derived from them include colorants such as inorganic pigments, examples being iron oxide, titanium oxide, Prussian Blue, and organic dyes, such as alizarin dyes, azo dyes and metal phthalocyanine dyes, and nutrients and trace nutrients, such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present. Additionally present may be foam-formers or defoamers.

Furthermore, the formulations and application forms derived from them may also comprise, as additional auxiliaries, stickers such as carboxymethylcellulose, natural and synthetic polymers in powder, granule or latex form, such as gum arabic, polyvinyl alcohol, polyvinyl acetate, and also natural phospholipids, such as cephalins and lecithins, and synthetic phospholipids. Further possible auxiliaries include mineral and vegetable oils.

There may possibly be further auxiliaries present in the formulations and the application forms derived from them. Examples of such additives include fragrances, protective colloids, binders, adhesives, thickeners, thixotropic substances, penetrants, retention promoters, stabilizers, sequestrants, complexing agents, humectants and spreaders. Generally speaking, the active compounds may be combined with any solid or liquid additive commonly used for formulation purposes.

Suitable retention promoters include all those substances which reduce the dynamic surface tension, such as dioctyl sulphosuccinate, or increase the viscoelasticity, such as hydroxypropylguar polymers, for example.

Suitable penetrants in the present context include all those substances which are typically used in order to enhance the penetration of active agrochemical compounds into plants. Penetrants in this context are defined in that, from the (generally aqueous) application liquor and/or from the spray coating, they are able to penetrate the cuticle of the plant and thereby increase the mobility of the active compounds in the cuticle. This property can be determined using the method described in the literature (Baur et al., 1997, Pesticide Science 51, 131-152). Examples include alcohol alkoxylates such as coconut fatty ethoxylate (10) or isotridecyl ethoxylate (12), fatty acid esters such as rapeseed or soybean oil methyl esters, fatty amine alkoxylates such as tallowamine ethoxylate (15), or ammonium and/or phosphonium salts such as ammonium sulphate or diammonium hydrogen phosphate, for example.

### Deposit Information

Samples of the Paenibacillus sp. strains of the invention have been deposited with the Agricultural Research Service Culture Collection located at the National Center for Agricultural Utilization Research, Agricultural Research Service, U.S. Department of Agriculture (NRRL), 1815 North University Street, Peoria, Ill. 61604, U.S.A., under the Budapest Treaty. Paenibacillus sp. strain NRRL B-50972 was deposited on Aug. 28, 2014. Paenibacillus sp. NRRL B-67129 was deposited on Sep. 1, 2015. Paenibacillus sp. NRRL B-67304 and Paenibacillus sp. NRRL B-67306 were both deposited on Jul. 22, 2016. Paenibacillus sp. NRRL B-67615 was deposited on May 3, 2018.

The Paenibacillus sp. strains have been deposited under conditions that assure that access to the culture will be available during the pendency of this patent application to one determined by the Commissioner of Patents and Trademarks to be entitled thereto under 37 C.F.R. § 1.14 and 35 U.S.C. § 122. However, it should be understood that the availability of a deposit does not constitute a license to practice the subject invention in derogation of patent rights granted by governmental action.

The following examples are given for purely illustrative and non-limiting purposes of the present invention.

### Examples

### Example 1. Antifungal Activity of Stabilized Paenibacillus Formulations

Paenibacillus sp. strain NRRL B-50972 was grown in a soy-based medium to produce a whole broth culture. The whole broth culture was then centrifuged and concentrated to generate a broth concentrate (BC). To evaluate the effect of various compounds as stabilizing agents samples of the Paenibacillus sp. strain NRRL B-50972 BC were taken and mixed with urea, guanidine hydrochloride, triethanolamine, or glycerol ethyl oxylate to a final concentration of 2% (i.e., 20 mg/mL) in a 60:40 (BC:water) dilution. The samples were divided into three groups which were kept at one of the following storage conditions: 1) ambient temperature (23° C.) for two weeks; 2) 40° C. for two weeks; or 3) 54° C. for two weeks. A 60:40 (BC:water) dilution of Paenibacillus sp. strain NRRL B-50972 BC without any stabilizing agent was included as a control in each group.

At the end of the two weeks of storage, the samples were applied to tomato plants at the concentrations shown in Table 2. The concentrations (i.e., application rates) are dilutions of the referenced treatment in water. After the various treatments had been applied, a solution containing an inoculum of Alternaria solani was sprayed on the plants. Several days after exposure to the inoculum of plant pathogen, each plant was scored for percent control of the pathogen relative to the untreated control plants. The disease incidence present with untreated control plants in each assay was between 75% and 100%. Treatments were evaluated with three or four replicates, and the average percent control and standard deviation were reported (see Table 2). 0% means an efficacy which corresponds to that of the untreated control, while an efficacy of 100% means that no disease is observed. A control treatment with SERENADE® ASO (Bacillus subtilis QST713), which is known to have activity against Alternaria solani, was included with each assay. In addition, a blank containing only urea diluted in water was applied to plants and was not observed to have any activity against Alternaria solani.

Samples of the 60:40 (BC:water) dilution of Paenibacillus sp. strain NRRL B-50972 BC mixed with urea, guanidine hydrochloride, or triethanolamine were also subjected to storage conditions of: 1) 4° C. for eight weeks; 2) ambient temperature (23° C.) for eight weeks; or 3) 40° C. for eight weeks. The samples were diluted in water and evaluated with the assay for activity against Alternaria solani in tomato plants as described above. The results of this second set of assays are presented in Table 3.

A third set of assays was conducted with the samples of 60:40 (BC:water) dilutions of Paenibacillus sp. strain NRRL B-50972 BC mixed with urea, guanidine hydrochloride, triethanolamine, or glycerol ethyl oxylate subjected to storage conditions of: 1) 4° C. for six months; or 2) ambient temperature (23° C.) for six months. The results from these assays are shown in Table 4.

Across the three assays, urea, guanidine hydrochloride, and triethanolamine each preserved the antifungal activity of the Paenibacillus sp. strain NRRL B-50972 BC while addition of glycerol ethyl oxylate had little effect. As expected, the stabilizing effect of the compounds was most pronounced in the samples subjected to the harshest storage conditions (i.e., 54° C. for two weeks shown in Table 2, 40° C. for eight weeks in Table 3, and ambient temperature (23° C.) for six months in Table 4).

TABLE 2
Antifungal activity of Paenibacillus sp. strain NRRL B-50972 broth
concentrate (BC) mixed with urea, guanidine HCl, triethanolamine, or
glycerol ethyl oxylate against Alternaria solani after two weeks
storage at ambient temperature (23° C.), 40° C., or 54° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 2.5 | 99 | 1 |
| subtilis QST713) | 1.25 | 98 | 1 |
| | 0.625 | 80 | 17 |

Antifungal Activity after Storage at Ambient Temperature for 2 Weeks

| | | | |
|---|---|---|---|
| Paenibacillus sp. strain NRRL | 2.5 | 99 | 1 |
| B-50972 BC | 1.25 | 96 | 1 |
| Paenibacillus sp. strain NRRL | 2.5 | 100 | 0 |
| B-50972 BC + Urea | 1.25 | 98 | 3 |
| Paenibacillus sp. strain NRRL | 2.5 | 100 | 1 |
| B-50972 BC + Guanidine HCl | 1.25 | 99 | 2 |
| Paenibacillus sp. strain NRRL | 2.5 | 99 | 1 |
| B-50972 BC + Triethanolamine | 1.25 | 96 | 1 |
| Paenibacillus sp. strain NRRL | 2.5 | 98 | 1 |
| B-50972 BC + Glycerol Ethyl | 1.25 | 89 | 0 |
| Oxylate | | | |

Antifungal Activity after Storage at 40° C. for 2 Weeks

| | | | |
|---|---|---|---|
| Paenibacillus sp. strain NRRL | 2.5 | 99 | 1 |
| B-50972 BC | 1.25 | 89 | 5 |
| Paenibacillus sp. strain NRRL | 2.5 | 99 | 1 |
| B-50972 BC + Urea | 1.25 | 96 | 2 |
| Paenibacillus sp. strain NRRL | 2.5 | 99 | 1 |
| B-50972 BC + Guanidine HCl | 1.25 | 98 | 3 |
| Paenibacillus sp. strain NRRL | 2.5 | 98 | 2 |
| B-50972 BC + Triethanolamine | 1.25 | 91 | 6 |
| Paenibacillus sp. strain NRRL | 2.5 | 95 | 0 |
| B-50972 BC + Glycerol Ethyl | 1.25 | 77 | 11 |
| Oxylate | | | |

Antifungal Activity after Storage at 54° C. for 2 Weeks

| | | | |
|---|---|---|---|
| Paenibacillus sp. strain NRRL | 2.5 | 86 | 3 |
| B-50972 BC | 1.25 | 54 | 12 |
| Paenibacillus sp. strain NRRL | 2.5 | 91 | 6 |
| B-50972 BC + Urea | 1.25 | 93 | 3 |
| Paenibacillus sp. strain NRRL | 2.5 | 99 | 2 |
| B-50972 BC + Guanidine HCl | 1.25 | 93 | 3 |
| Paenibacillus sp. strain NRRL | 2.5 | 97 | 0 |
| B-50972 BC + Triethanolamine | 1.25 | 95 | 0 |
| Paenibacillus sp. strain NRRL | 2.5 | 79 | 5 |
| B-50972 BC + Glycerol Ethyl | 1.25 | 59 | 34 |
| Oxylate | | | |

TABLE 3
Antifungal activity of Paenibacillus sp. strain NRRL
B-50972 BC mixed with urea, guanidine HCl, or triethanolamine
against Alternaria solani after eight weeks storage
at 4° C., ambient temperature (23° C.), or 40° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 2.5 | 69 | 3 |
| subtilis QST713) | 1.25 | 45 | 7 |
| | 0.625 | 10 | 17 |

Antifungal Activity after Storage at 4° C. for 8 Weeks

| | | | |
|---|---|---|---|
| Paenibacillus sp. strain NRRL | 2.5 | 82 | 0 |
| B-50972 BC | 1.25 | 63 | 9 |
| Paenibacillus sp. strain NRRL | 2.5 | 82 | 6 |
| B-50972 BC + Urea | 1.25 | 61 | 14 |
| Paenibacillus sp. strain NRRL | 2.5 | 80 | 3 |
| B-50972 BC + Guanidine HCl | 1.25 | 63 | 21 |
| Paenibacillus sp. strain NRRL | 2.5 | 78 | 7 |
| B-50972 BC + Triethanolamine | 1.25 | 63 | 9 |

Antifungal Activity after Storage at Ambient Temperature for 8 Weeks

| | | | |
|---|---|---|---|
| Paenibacillus sp. strain NRRL | 2.5 | 71 | 16 |
| B-50972 BC | 1.25 | 73 | 9 |
| Paenibacillus sp. strain NRRL | 2.5 | 80 | 9 |
| B-50972 BC + Urea | 1.25 | 76 | 20 |
| Paenibacillus sp. strain NRRL | 2.5 | 90 | 7 |
| B-50972 BC + Guanidine HCl | 1.25 | 76 | 6 |
| Paenibacillus sp. strain NRRL | 2.5 | 73 | 17 |
| B-50972 BC + Triethanolamine | 1.25 | 69 | 15 |

Antifungal Activity after Storage at 40° C. for 8 Weeks

| | | | |
|---|---|---|---|
| Paenibacillus sp. strain NRRL | 2.5 | 61 | 7 |
| B-50972 BC | 1.25 | 53 | 0 |
| Paenibacillus sp. strain NRRL | 2.5 | 86 | 3 |
| B-50972 BC + Urea | 1.25 | 61 | 7 |
| Paenibacillus sp. strain NRRL | 2.5 | 90 | 3 |
| B-50972 BC + Guanidine HCl | 1.25 | 67 | 15 |
| Paenibacillus sp. strain NRRL | 2.5 | 53 | 12 |
| B-50972 BC + Triethanolamine | 1.25 | 49 | 7 |

TABLE 4
Antifungal activity of Paenibacillus sp. strain NRRL B-50972
BC mixed with urea, guanidine HCl, triethanolamine, or glycerol
ethyl oxylate against Alternaria solani after six months
storage at 4° C. or ambient temperature (23° C.).

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 5.0 | 89 | 5 |
| subtilis QST713) | 2.5 | 89 | 5 |
| | 1.25 | 77 | 8 |
| | 0.625 | 40 | 30 |

Antifungal Activity after Storage at 4° C. for 6 Months

| | | | |
|---|---|---|---|
| Paenibacillus sp. strain NRRL | 2.5 | 97 | 3 |
| B-50972 BC | 1.25 | 84 | 11 |
| Paenibacillus sp. strain NRRL | 2.5 | 97 | 3 |
| B-50972 BC + Urea | 1.25 | 88 | 3 |
| Paenibacillus sp. strain NRRL | 2.5 | 99 | 2 |
| B-50972 BC + Guanidine HCl | 1.25 | 91 | 3 |
| Paenibacillus sp. strain NRRL | 2.5 | 97 | 3 |
| B-50972 BC + Triethanolamine | 1.25 | 86 | 3 |
| Paenibacillus sp. strain NRRL | 2.5 | 97 | 0 |
| B-50972 BC + Glycerol Ethyl | 1.25 | 92 | 7 |
| Oxylate | | | |

Antifungal Activity after Storage at Ambient Temperature for 6 Months

| | | | |
|---|---|---|---|
| Paenibacillus sp. strain NRRL | 2.5 | 88 | 8 |
| B-50972 BC | 1.25 | 67 | 8 |
| Paenibacillus sp. strain NRRL | 2.5 | 98 | 2 |
| B-50972 BC + Urea | 1.25 | 91 | 3 |
| Paenibacillus sp. strain NRRL | 2.5 | 98 | 2 |
| B-50972 BC + Guanidine HCl | 1.25 | 89 | 0 |
| Paenibacillus sp. strain NRRL | 2.5 | 92 | 4 |
| B-50972 BC + Triethanolamine | 1.25 | 86 | 8 |
| Paenibacillus sp. strain NRRL | 2.5 | 77 | 8 |
| B-50972 BC + Glycerol Ethyl | 1.25 | 77 | 3 |
| Oxylate | | | |

### Example 2. Quantification of Fusaricidin a in Stabilized Paenibacillus Formulations

Paenibacillus cells are known to produce several antifungal compounds including fusaricidin A. To determine the effect of the stabilizing agents on the levels of fusaricidin A in the samples of Paenibacillus sp. strain NRRL B-50972 BC mixed with urea, guanidine hydrochloride, triethanolamine, or glycerol ethyl oxylate analyzed in Example 1 the samples were extracted with organic solvent to produce cell extracts.

Relative levels of fusaricidin A in these extracts were quantified with a chromatographic method using high-performance liquid chromatography/mass spectrometry time-of-flight (HPLC/MS TOF) as follows: Column: YMC™ Basic 4.6×250 mm, 5 μm; Water (0.1% FA) and Acetonitrile (0.1% formic acid (FA)); Gradient (% B): 0-9 min 28-30%; 9-14 min 30-33%; 14-34 min 33-50%; Wash.

A standard sample stored at −80° C. containing a whole broth culture of Paenibacillus sp. strain NRRL B-50972 was run with each HPLC/MS-TOF analysis, and the peak area of the fusaricidin A from this standard sample was used as the basis to normalize the peak areas in the remaining samples and calculate the relative amounts reported.

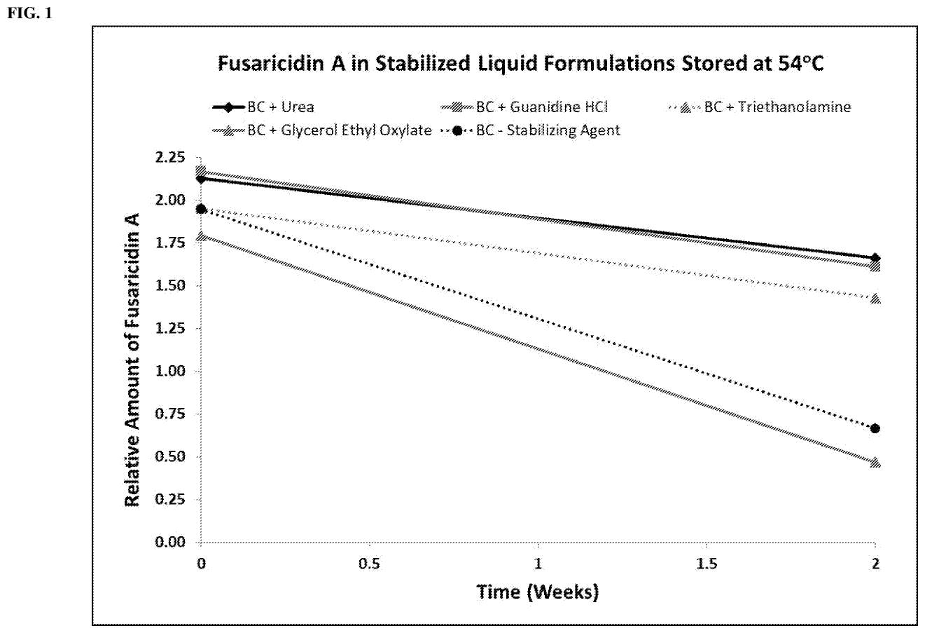

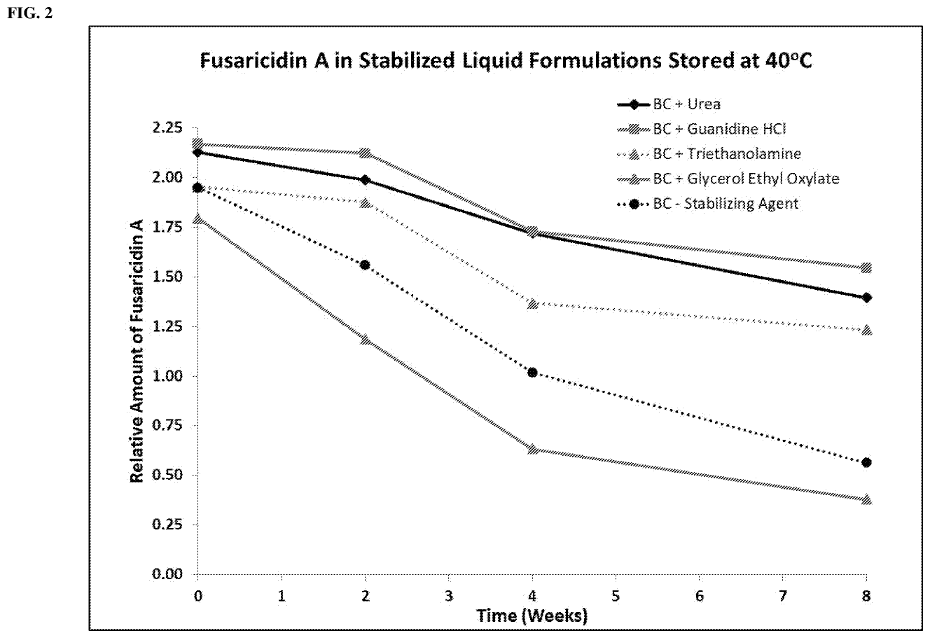

The relative amounts of fusaricidin A in the samples of Paenibacillus sp. strain NRRL B-50972 BC with and without stabilizing agents stored at 54° C. for two weeks or at 40° C. for eight weeks are shown in FIG. 1 and FIG. 2, respectively. For the Paenibacillus sp. strain NRRL B-50972 BC stored at 40° C., samples were taken and analyzed from 0 weeks, 2 weeks, 4 weeks, and 8 weeks of storage.

Urea, guanidine hydrochloride, and triethanolamine inhibited the degradation of fusaricidin A in the Paenibacillus sp. strain NRRL B-50972 BC samples. In contrast, addition of glycerol ethyl oxylate had little effect on the stabilization of fusaricidin A in these samples. Relative quantification of fusaricidin A in the Paenibacillus sp. strain NRRL B-50972 BC samples stored at ambient temperature (23° C.) for six months confirmed the trend observed in FIGS. 1 and 2.

These results agree with the data presented in Tables 2-4 where urea, guanidine hydrochloride, and triethanolamine preserved antifungal activity against Alternaria solani after storage of the Paenibacillus sp. strain NRRL B-50972 BC over extended periods of time and/or at elevated temperatures whereas glycerol ethyl oxylate had little effect.

### Example 3. Evaluation of Additional Stabilized Paenibacillus Formulations

Additional stabilized formulations of Paenibacillus sp. strain NRRL B-50972 BC containing one of several candidate stabilizing agents were prepared and evaluated for antifungal activity against Alternaria solani as described in Example 1. The candidate stabilizing agents were added to a 70:30 (BC:water) dilution of the Paenibacillus sp. strain NRRL B-50972 BC to final concentrations of 2% or 0.5%. The stabilized formulations were then stored at 54° C. for two weeks or at 40° C. for eight weeks and compared to the Paenibacillus sp. strain NRRL B-50972 BC without any stabilizing agent.

Assays determining the antifungal activity of the different stabilized formulations were performed as described in Example 1. Activity against either Alternaria solani or Botrytis cinerea was measured at the application rates indicated in Tables 5-8. The concentrations (i.e., application rates) are dilutions of the referenced treatment in water. 0% means an efficacy which corresponds to that of the untreated control, while an efficacy of 100% means that no disease is observed.

Along with urea, betaine hydrochloride showed a robust effect in stabilizing the antifungal activity of the Paenibacillus sp. strain NRRL B-50972 BC against both Alternaria solani and Botrytis cinerea. The stabilizing effect of potassium phosphate (dibasic) was less than that of urea and betaine hydrochloride. Several other candidate stabilizing agents had no significant effect on the antifungal activity of the Paenibacillus sp. strain NRRL B-50972 BC.

TABLE 5
Antifungal activity of Paenibacillus sp. strain NRRL
B-50972 broth concentrate (BC) mixed with urea, betaine
HCl, or potassium phosphate (dibasic) against Alternaria solani
after two weeks storage at 54° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| 2% Urea Blank | 1.25 | 0 | 0 |
| 2% Betaine HCl Blank | 1.25 | 0 | 0 |
| 2% Potassium Phosphate (Dibasic) | 1.25 | 0 | 0 |
| Blank | | | |
| SERENADE ® ASO (Bacillus | 5 | 88 | 3 |
| subtilis QST713) | 2.5 | 67 | 12 |
| | 1.25 | 47 | 12 |
| | 0.625 | 25 | 15 |

Initial Antifungal Activity Before Storage

| | | | |
|---|---|---|---|
| Paenibacillus sp. strain NRRL | 1.25 | 87 | 8 |
| B-50972 BC | 0.625 | 57 | 15 |

Antifungal Activity after Storage at 54° C. for 2 Weeks

| | | | |
|---|---|---|---|
| Paenibacillus sp. strain NRRL | 1.25 | 3 | 6 |
| B-50972 BC | 0.625 | 20 | 35 |
| Paenibacillus sp. strain NRRL | 1.25 | 78 | 8 |
| B-50972 BC + 2% Urea | 0.625 | 57 | 15 |
| Paenibacillus sp. strain NRRL | 1.25 | 57 | 21 |
| B-50972 BC + 0.5% Urea | 0.625 | 10 | 13 |
| Paenibacillus sp. strain NRRL | 1.25 | 63 | 6 |
| B-50972 BC + 2% Betaine HCl | 0.625 | 50 | 39 |
| Paenibacillus sp. strain NRRL | 1.25 | 42 | 18 |
| B-50972 BC + 0.5% Betaine HCl | 0.625 | 0 | 0 |
| Paenibacillus sp. strain NRRL | 1.25 | 68 | 18 |
| B-50972 BC + 2% Potassium | 0.625 | 63 | 6 |
| Phosphate (Dibasic) | | | |
| Paenibacillus sp. strain NRRL | 1.25 | 5 | 5 |
| B-50972 BC + 0.5% Potassium | 0.625 | 0 | 0 |
| Phosphate (Dibasic) | | | |

TABLE 6
Antifungal activity of Paenibacillus sp. strain NRRL
B-50972 broth concentrate (BC) mixed with urea, betaine
HCl, or potassium phosphate (dibasic) against Botrytis cinerea
after two weeks storage at 54° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| 2% Urea Blank | 1.25 | 0 | 0 |
| 2% Betaine HCl Blank | 1.25 | 0 | 0 |
| 2% Potassium Phosphate (Dibasic) | 1.25 | 0 | 0 |
| Blank | | | |
| SERENADE ® ASO (Bacillus | 5 | 100 | 0 |
| subtilis QST713) | 2.5 | 98 | 2 |
| | 1.25 | 80 | 21 |
| | 0.625 | 53 | 41 |

Initial Antifungal Activity Before Storage

| | | | |
|---|---|---|---|
| Paenibacillus sp. strain NRRL | 1.25 | 81 | 18 |
| B-50972 BC | 0.625 | 26 | 20 |

Antifungal Activity after Storage at 54° C. for 2 Weeks

| | | | |
|---|---|---|---|
| Paenibacillus sp. strain NRRL | 1.25 | 21 | 23 |
| B-50972 BC | 0.625 | 6 | 6 |
| Paenibacillus sp. strain NRRL | 1.25 | 61 | 5 |
| B-50972 BC + 2% Urea | 0.625 | 23 | 5 |
| Paenibacillus sp. strain NRRL | 1.25 | 59 | 9 |
| B-50972 BC + 2% Betaine HCl | 0.625 | 25 | 33 |
| Paenibacillus sp. strain NRRL | 1.25 | 39 | 21 |
| B-50972 BC + 2% Potassium | 0.625 | 9 | 11 |
| Phosphate (Dibasic) | | | |

TABLE 7
Antifungal activity of Paenibacillus sp. strain NRRL
B-50972 broth concentrate (BC) mixed with urea, betaine
HCl, or potassium phosphate (dibasic) against Alternaria solani
after eight weeks storage at 40° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 5 | 88 | 6 |
| subtilis QST713) | 2.5 | 78 | 9 |
| | 1.25 | 65 | 20 |
| | 0.625 | 53 | 20 |

Initial Antifungal Activity Before Storage

| | | | |
|---|---|---|---|
| Paenibacillus sp. strain NRRL | 1.25 | 86 | 9 |
| B-50972 BC | 0.625 | 82 | 10 |

Antifungal Activity after Storage at 40° C. for 8 Weeks

| | | | |
|---|---|---|---|
| Paenibacillus sp. strain NRRL | 1.25 | 69 | 7 |
| B-50972 BC | 0.625 | 41 | 12 |
| Paenibacillus sp. strain NRRL | 1.25 | 85 | 8 |
| B-50972 BC + 2% Urea | 0.625 | 80 | 7 |
| Paenibacillus sp. strain NRRL | 1.25 | 73 | 3 |
| B-50972 BC + 0.5% Urea | 0.625 | 47 | 33 |
| Paenibacillus sp. strain NRRL | 1.25 | 83 | 12 |
| B-50972 BC + 2% Betaine HCl | 0.625 | 83 | 13 |
| Paenibacillus sp. strain NRRL | 1.25 | 84 | 7 |
| B-50972 BC + 0.5% Betaine HCl | 0.625 | 45 | 7 |
| Paenibacillus sp. strain NRRL | 1.25 | 61 | 7 |
| B-50972 BC + 2% Potassium | 0.625 | 55 | 15 |
| Phosphate (Dibasic) | | | |
| Paenibacillus sp. strain NRRL | 1.25 | 41 | 12 |
| B-50972 BC + 0.5% Potassium | 0.625 | 51 | 21 |
| Phosphate (Dibasic) | | | |

TABLE 8
Antifungal activity of Paenibacillus sp. strain NRRL
B-50972 broth concentrate (BC) mixed with urea, betaine
HCl, or potassium phosphate (dibasic) against Botrytis cinerea
after eight weeks storage at 40° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 5 | 100 | 1 |
| subtilis QST713) | 2.5 | 98 | 2 |
| | 1.25 | 90 | 13 |
| | 0.625 | 24 | 20 |

Initial Antifungal Activity Before Storage

| | | | |
|---|---|---|---|
| Paenibacillus sp. strain NRRL | 1.25 | 81 | 12 |
| B-50972 BC | 0.625 | 58 | 26 |

Antifungal Activity after Storage at 40° C. for 8 Weeks

| | | | |
|---|---|---|---|
| Paenibacillus sp. strain NRRL | 1.25 | 3 | 2 |
| B-50972 BC | 0.625 | 3 | 5 |
| Paenibacillus sp. strain NRRL | 1.25 | 71 | 9 |
| B-50972 BC + 2% Urea | 0.625 | 21 | 22 |
| Paenibacillus sp. strain NRRL | 1.25 | 64 | 9 |
| B-50972 BC + 2% Betaine HCl | 0.625 | 46 | 33 |
| Paenibacillus sp. strain NRRL | 1.25 | 34 | 23 |
| B-50972 BC + 2% Potassium | 0.625 | 23 | 15 |
| Phosphate (Dibasic) | | | |

### Example 4. Quantification of Fusaricidin a in Additional Stabilized Paenibacillus Formulations

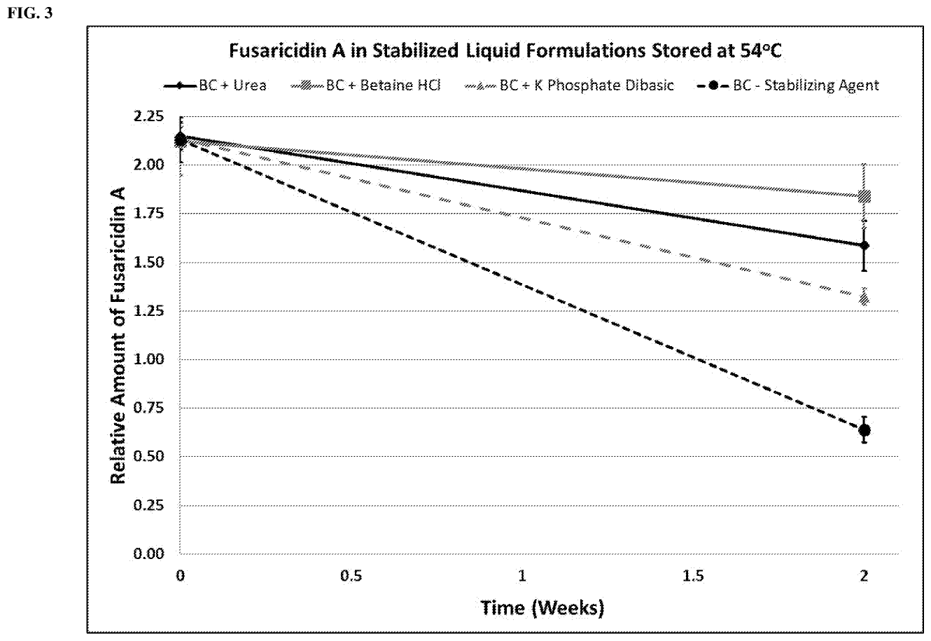

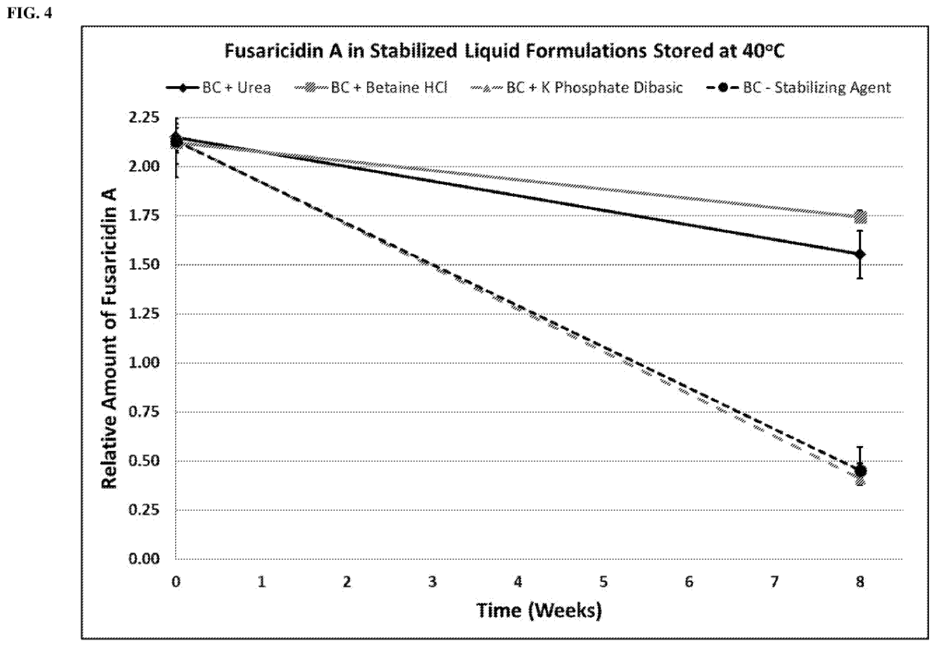

The quantitative method described in Example 2 was used to determine the relative amounts of fusaricidin A present in the samples prepared in Example 3. The relative amounts of fusaricidin A in these additional samples of Paenibacillus sp. strain NRRL B-50972 BC with and without stabilizing agents after storage at 54° C. for two weeks or storage at 40° C. for eight weeks are presented in FIG. 3 and FIG. 4, respectively.

Urea and betaine hydrochloride preserved the fusaricidin A in the Paenibacillus sp. strain NRRL B-50972 BC samples whereas potassium phosphate (dibasic) had less of an effect in stabilizing fusaricidin A in these samples. The samples of Paenibacillus sp. strain NRRL B-50972 BC mixed with candidate stabilizing agents that did not preserve antifungal activity were also analyzed. Degradation of fusaricidin A in these samples was similar to that in the Paenibacillus sp. strain NRRL B-50972 BC without any stabilizing agent.

These results agree with the data presented in Tables 5-8 where addition of urea and betaine hydrochloride maintained antifungal activity against Alternaria solani and Botrytis cinerea after storage of the Paenibacillus sp. strain NRRL B-50972 BC at 54° C. for two weeks or at 40° C. for eight weeks while potassium phosphate (dibasic) had less of a stabilizing effect.

### Example 5. Evaluation of Stabilized Paenibacillus Formulations with Potassium Phosphate (Monobasic), Potassium Phosphate (Dibasic) or Potassium Citrate (Tribasic)

Several closely related compounds were tested for their potential to stabilize Paenibacillus sp. strain NRRL B-50972 BC after storage at 54° C. for two weeks. The samples were prepared and analyzed as described in Example 3 and contained urea, potassium phosphate (monobasic), potassium phosphate (dibasic) or potassium citrate (tribasic) at 2% in the 70:30 (BC:water) dilution of the Paenibacillus sp. strain NRRL B-50972 BC. A sample of Paenibacillus sp. strain NRRL B-50972 whole broth (WB) prior to centrifugation and concentration was included as a control. After storage at 54° C. for two weeks the antifungal activity in the WB control sample had been reduced so that the disease present on plants treated with WB was not distinguishable from that of untreated plants (data not shown).

The antifungal activity of the Paenibacillus sp. strain NRRL B-50972 BC samples mixed with urea, potassium phosphate (monobasic), potassium phosphate (dibasic) or potassium citrate (tribasic) after storage at 54° C. for two weeks is shown in Table 9. The concentrations (i.e., application rates) are dilutions of the referenced treatment in water. 0% means an efficacy which corresponds to that of the untreated control, while an efficacy of 100% means that no disease is observed. Surprisingly, potassium phosphate (dibasic) stabilized the antifungal activity whereas potassium phosphate (monobasic) did not. Potassium citrate (tribasic) also demonstrated a stabilizing effect on the Paenibacillus sp. strain NRRL B-50972 BC.

TABLE 9
Antifungal activity of Paenibacillus sp. strain NRRL B-50972 broth
concentrate (BC) mixed with urea, potassium phosphate (monobasic),
potassium phosphate (dibasic), or potassium citrate (tribasic) against
Alternaria solani after two weeks storage at 54° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 5 | 90 | 5 |
| subtilis QST713) | 2.5 | 63 | 12 |
| | 1.25 | 70 | 9 |

Antifungal Activity after Storage at 54° C. for 2 Weeks

| | | | |
|---|---|---|---|
| Paenibacillus sp. strain NRRL | 1.25 | 82 | 10 |
| B-50972 BC + 2% Urea | 0.625 | 53 | 38 |
| Paenibacillus sp. strain NRRL | 1.25 | 28 | 20 |
| B-50972 BC + 2% Potassium | 0.625 | 8 | 14 |
| Phosphate (Monobasic) | | | |
| Paenibacillus sp. strain NRRL | 1.25 | 80 | 9 |
| B-50972 BC + 2% Potassium | 0.625 | 77 | 3 |
| Phosphate (Dibasic) | | | |
| Paenibacillus sp. strain NRRL | 1.25 | 65 | 22 |
| B-50972 BC + 2% Potassium | 0.625 | 57 | 15 |
| Citrate (Tribasic) | | | |

### Example 5. Determination of the Effect of Solvents on Paenibacillus Formulations

Paenibacillus sp. strain NRRL B-50972 was grown in a soy-based medium to produce a whole broth culture. The whole broth culture was then centrifuged and concentrated to generate a broth concentrate (BC). A 50:50 dilution of the Paenibacillus sp. strain NRRL B-50972 BC in water or propylene glycol was prepared and subjected to analysis of antifungal activity and relative fusaricidin A levels as described in Examples 1 and 2. The concentrations (i.e., application rates) are dilutions of the referenced treatment in water. 0% means an efficacy which corresponds to that of the untreated control, while an efficacy of 100% means that no disease is observed.

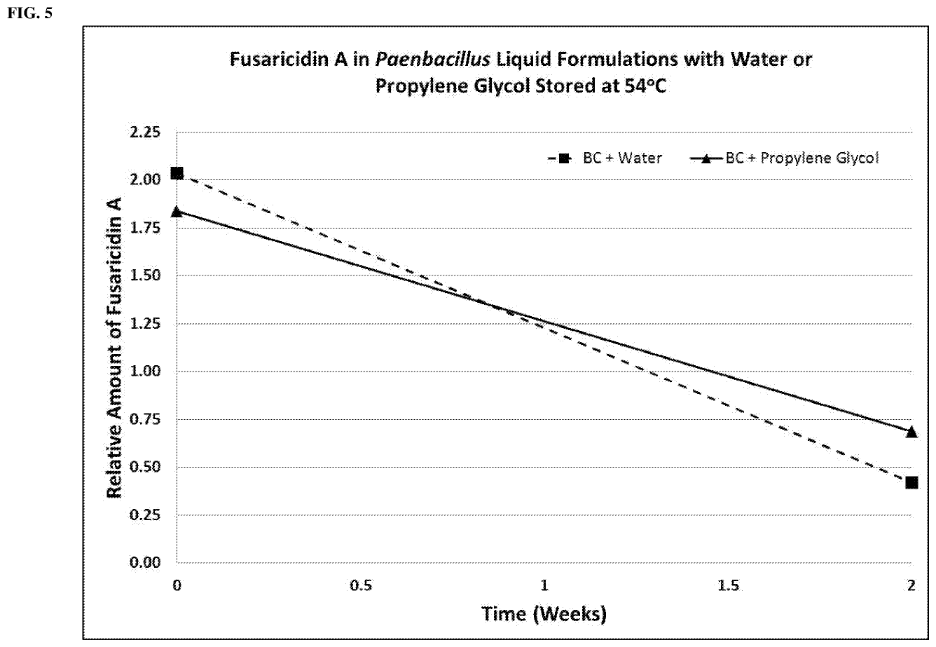

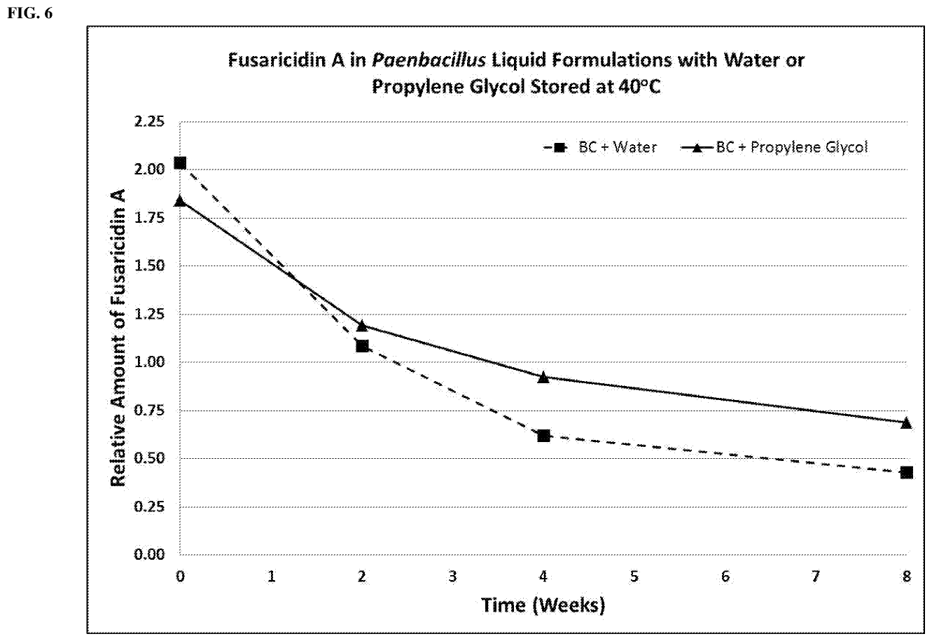

Dilution in propylene glycol preserved the antifungal activity of the Paenibacillus sp. strain NRRL B-50972 BC to a greater degree than did dilution in water after storage for two weeks at 54° C. or for two, four, or eight weeks at 40° C. (compare Tables 10 and 11). In addition, the Paenibacillus sp. strain NRRL B-50972 BC diluted in propylene glycol had a slower rate of degradation of fusaricidin A after storage for two weeks at 54° C. or for two, four, or eight weeks at 40° C. than did the Paenibacillus sp. strain NRRL B-50972 BC diluted in water (compare FIGS. 5 and 6).

TABLE 10
Antifungal activity of Paenibacillus sp. strain NRRL B-50972
broth concentrate (BC) in a 50:50 dilution with water against
Alternaria solani after two weeks storage at 54°
C. or after two, four, or eight weeks storage at 40° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 5 | 100 | 0 |
| subtilis QST713) | 2.5 | 90 | 0 |
| | 1.25 | 72 | 3 |
| | 0.625 | 67 | 8 |

Antifungal Activity after Storage at 54° C. for 2 Weeks

| | | | |
|---|---|---|---|
| Paenibacillus sp. strain NRRL | 5 | 95 | 0 |
| B-50972 BC + Water (0 weeks) | 2.5 | 93 | 3 |
| | 1.25 | 88 | 3 |
| Paenibacillus sp. strain NRRL | 5 | 62 | 12 |
| B-50972 BC + Water (2 weeks) | 2.5 | 40 | 15 |
| | 1.25 | 10 | 11 |

Antifungal Activity after Storage at 40° C. for 2, 4, or 8 Weeks

| | | | |
|---|---|---|---|
| Paenibacillus sp. strain NRRL | 5 | 95 | 0 |
| B-50972 BC + Water (0 weeks) | 2.5 | 93 | 3 |
| | 1.25 | 88 | 3 |
| Paenibacillus sp. strain NRRL | 5 | 95 | 0 |
| B-50972 BC + Water (2 weeks) | 2.5 | 90 | 5 |
| | 1.25 | 64 | 14 |
| Paenibacillus sp. strain NRRL | 5 | 93 | 3 |
| B-50972 BC + Water (4 weeks) | 2.5 | 66 | 6 |
| | 1.25 | 15 | 13 |
| Paenibacillus sp. strain NRRL | 5 | 66 | 22 |
| B-50972 BC + Water (8 weeks) | 2.5 | 53 | 19 |
| | 1.25 | 13 | 22 |

TABLE 11
Antifungal activity of Paenibacillus sp. strain NRRL B-50972
broth concentrate (BC) in a 50:50 dilution with propylene glycol
against Alternaria solani after two weeks storage at 54°
C. or after two, four, or eight weeks storage at 40° C.

| | | | |
|---|---|---|---|
| | | Average | |
| | Concentration | Efficacy | Standard |
| Treatment | (%) | (%) | Deviation |

Antifungal Activity of Controls

| | | | |
|---|---|---|---|
| Untreated Control | | 0 | — |
| SERENADE ® ASO (Bacillus | 5 | 96 | 2 |
| subtilis QST713) | 2.5 | 83 | 12 |
| | 1.25 | 68 | 8 |
| | 0.625 | 40 | 26 |

Antifungal Activity after Storage at 54° C. for 2 Weeks

| | | | |
|---|---|---|---|
| Paenibacillus sp. strain NRRL | 5 | 93 | 3 |
| B-50972 BC + Propylene Glycol | 2.5 | 88 | 3 |
| (0 weeks) | 1.25 | 80 | 0 |
| Paenibacillus sp. strain NRRL | 5 | 88 | 3 |
| B-50972 BC + Propylene Glycol | 2.5 | 52 | 36 |
| (2 weeks) | 1.25 | 50 | 10 |

Antifungal Activity after Storage at 40° C. for 2, 4, or 8 Weeks

| | | | |
|---|---|---|---|
| Paenibacillus sp. strain NRRL | 5 | 93 | 3 |
| B-50972 BC + Propylene Glycol | 2.5 | 88 | 3 |
| (0 weeks) | 1.25 | 80 | 0 |
| Paenibacillus sp. strain NRRL | 5 | 90 | 5 |
| B-50972 BC + Propylene Glycol | 2.5 | 83 | 8 |
| (2 weeks) | 1.25 | 50 | 31 |
| Paenibacillus sp. strain NRRL | 5 | 87 | 6 |
| B-50972 BC + Propylene Glycol | 2.5 | 58 | 33 |
| (4 weeks) | 1.25 | 57 | 15 |
| Paenibacillus sp. strain NRRL | 5 | 77 | 8 |
| B-50972 BC + Propylene Glycol | 2.5 | 47 | 26 |
| (8 weeks) | 1.25 | 30 | 26 |

### Example 6. Improvement in Physical Properties of Stabilized Paenibacillus Formulations

Paenibacillus sp. strain NRRL B-50972 BC was prepared and diluted with a mixture of propylene glycol and water. A portion of the diluted Paenibacillus sp. strain NRRL B-50972 BC was mixed with 2% urea and the remainder contained no urea.

A formulation is considered to be physically stable if little or no syneresis or sedimentation of the formulation components is observed for a prolonged period of time. The longer it takes for syneresis or sedimentation to occur, the more physically stable is the formulation. A formulation is considered to be physically stable if little or no syneresis or sedimentation is observed after 2 weeks at 54° C.

Samples of the diluted Paenibacillus sp. strain NRRL B-50972 BC with and without urea were stored for 2 weeks at 54° C. No syneresis or sedimentation of the samples was observed prior to storage. Photographs of the samples after storage show that the diluted Paenibacillus sp. strain NRRL B-50972 BC with urea experienced little or no syneresis or sedimentation. In contrast, the photograph of the diluted Paenibacillus sp. strain NRRL B-50972 BC without urea shows separation of the formulation into distinct layers indicating syneresis and sedimentation. The results demonstrate the improvement in the physically stability of the Paenibacillus sp. strain NRRL B-50972 BC resulting from addition of urea.

### Example 7. Relative Fusaricidin a Levels and Antifungal Activity from Stabilized Paenibacillus Formulation after Two Years at 23° C.

Broth concentrates (BC) of Paenibacillus sp. strain NRRL B-50972 were prepared with and without 2% urea, and their antifungal activities against Alternaria solani were measured as described in Example 1 after two years of storage at 23° C. The concentrations (i.e., application rates) are dilutions of the referenced treatment in water. 0% means an efficacy which corresponds to that of the untreated control, while an efficacy of 100% means that no disease is observed. Relative levels of fusaricidin A in the broth concentrates were also determined as outlined in Example 2.

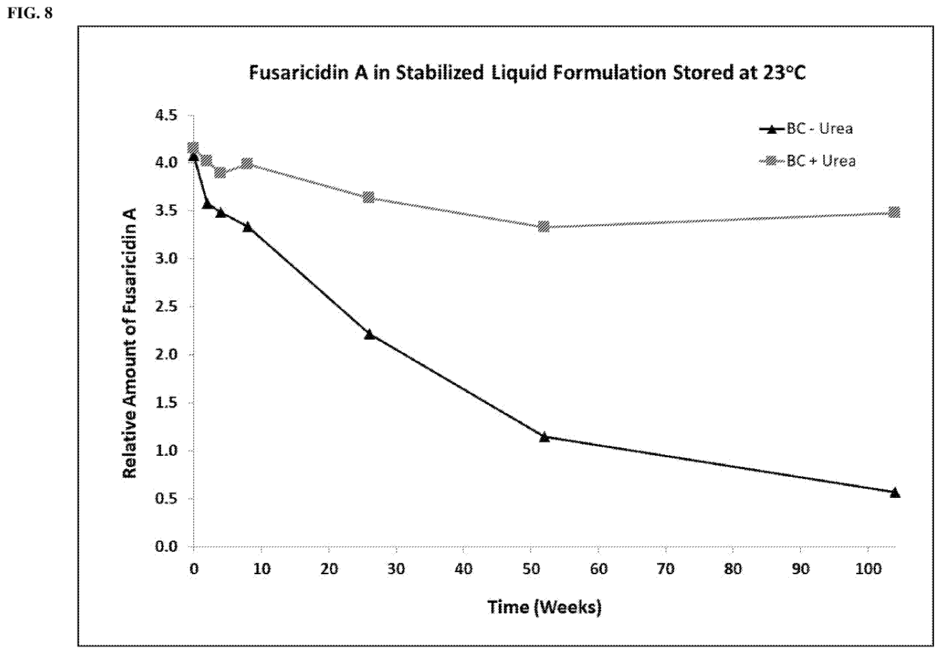

The antifungal activity of Paenibacillus sp. strain NRRL B-50972 BC containing 2% urea remained at about 90% even after two years of storage at 23° C. whereas the antifungal activity of Paenibacillus sp. strain NRRL B-50972 BC without urea dropped to less than half that level (see Table 12). Similarly, fusaricidin A levels in the Paenibacillus sp. strain NRRL B-50972 BC containing 2% urea remained relatively constant over two years of storage at 23° C. while fusaricidin A levels in the Paenibacillus sp. strain NRRL B-50972 BC without urea dropped to about one fourth the initial levels over this same period of storage (see FIG. 8).

### Example 8. Relative Fusaricidin a Levels and Antifungal Activity from Stabilized Formulations with Various Paenibacillus sp. Strains

Paenibacillus sp. strain NRRL B-50972 is related to Paenibacillus sp. strain NRRL B-67304, Paenibacillus sp. strain NRRL B-67306, and Paenibacillus sp. strain NRRL B-67615 as described in U.S. Patent Application No. 62/671,067. Paenibacillus sp. strain NRRL B-50972 is the parental strain from which each of the other strains were derived by multiple rounds of mutagenesis and screening for increased production of fusaricidins and fusaricidin-like compounds and decreased viscosity in liquid culture. Paenibacillus sp. strain NRRL B-67304, Paenibacillus sp. strain NRRL B-67306, and Paenibacillus sp. strain NRRL B-67615 each have mutations in the degU and/or degS genes that result in a non-mucoid colony morphology on solid culture medium and decreased viscosity in liquid cultures.

Broth concentrates of Paenibacillus sp. NRRL B-50972, Paenibacillus sp. strain NRRL B-67304, and Paenibacillus sp. strain NRRL B-67306 were prepared with urea, and their antifungal activities against Alternaria solani were measured as described in Example 1 after eight weeks of storage at 40° C. and after one year of storage at 23° C. The concentrations (i.e., application rates) are dilutions of the referenced treatment in water. 0% means an efficacy which corresponds to that of the untreated control, while an efficacy of 100% means that no disease is observed. Relative levels of fusaricidin A in the broth concentrates were also determined as outlined in Example 2.

The antifungal activity of Paenibacillus sp. strain NRRL B-50972 BC without urea was not significantly different from untreated control after eight weeks of storage at 40° C. whereas the antifungal activity of Paenibacillus sp. NRRL B-50972 BC, Paenibacillus sp. strain NRRL B-67304 BC, and Paenibacillus sp. strain NRRL B-67306 BC each mixed with urea remained at between 79% and 89% when applied at 0.625% or 0.312% to plants (see Table 13). Similar results were observed with the broth concentrates stored for one year at 23° C. except that the antifungal activity of the Paenibacillus sp. strain NRRL B-50972 BC without urea was detectable at low levels above untreated control under these conditions (see Table 14).

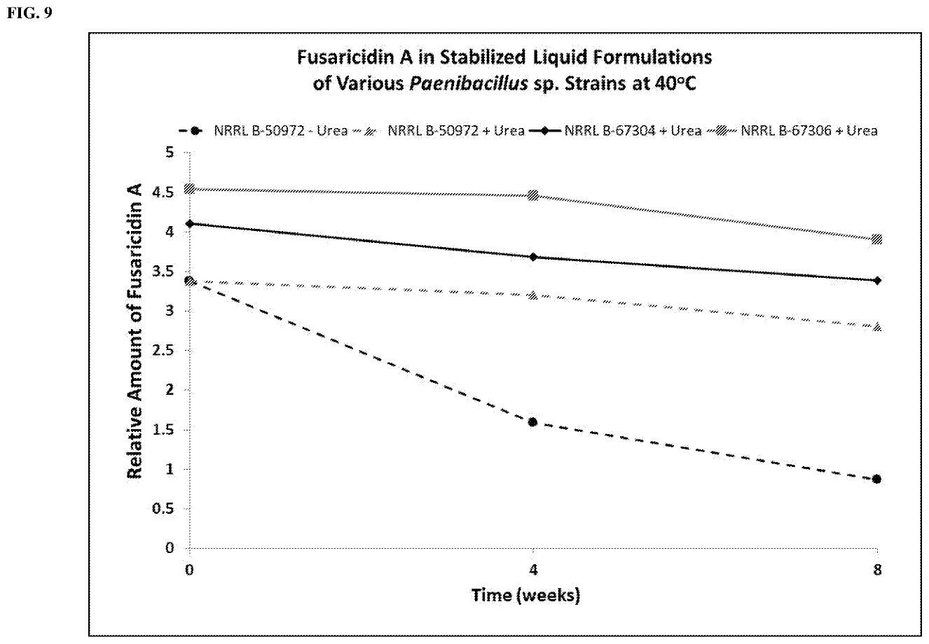

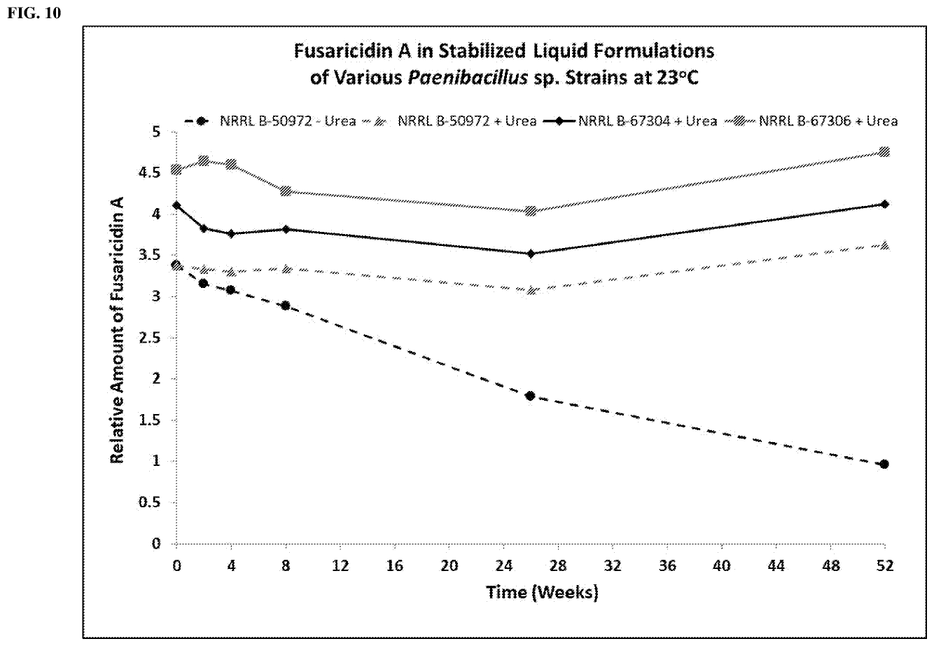

Fusaricidin A levels in the Paenibacillus sp. NRRL B-50972 BC, Paenibacillus sp. strain NRRL B-67304 BC, and Paenibacillus sp. strain NRRL B-67306 BC each mixed with urea remained relatively constant after eight weeks of storage at 40° C. and after one year of storage at 23° C. while fusaricidin A levels in the Paenibacillus sp. strain NRRL B-50972 BC without urea dropped to about one third the initial levels over these same periods of storage (see FIGS. 9 and 10).

TABLE 12
Antifungal activity of Paenibacillus sp. strain NRRL B-50972
broth concentrate (BC) with and without urea against Alternaria solani
after two years of storage at 23° C.

| | | | |
|---|---|---|---|
| | | Average | |
| | Concentration | Efficacy | Standard |
| Treatment | (%) | (%) | Error |

Antifungal Activity of Controls

| | | | |
|---|---|---|---|
| Untreated Control | | 0 | — |
| SERENADE ® ASO (Bacillus | 2.5 | 93 | 3 |
| subtilis QST713) | 0.625 | 88 | 4 |

Antifungal Activity after Storage at 23° C. for 2 Years

| | | | |
|---|---|---|---|
| Paenibacillus sp. strain NRRL | 2.5 | 40 | 20 |
| B-50972 BC | 0.625 | 19 | 19 |
| Paenibacillus sp. strain NRRL | 2.5 | 88 | 4 |
| B-50972 BC + 2% Urea | 0.625 | 93 | 3 |

TABLE 13
Antifungal activity of broth concentrate (BC) from various
Paenibacillus sp. strains stabilized with urea against
Alternaria solani after eight weeks of storage at 40° C.

| | | | |
|---|---|---|---|
| | | Average | |
| | Concentration | Efficacy | Standard |
| Treatment | (%) | (%) | Error |

Antifungal Activity of Controls

| | | | |
|---|---|---|---|
| Untreated Control | | 0 | — |
| SERENADE ® ASO (Bacillus | 5 | 82 | 5 |
| subtilis QST713) | 2.5 | 68 | 0 |
| | 1.25 | 50 | 8 |

Antifungal Activity after Storage at 40° C. for 8 Weeks

| | | | |
|---|---|---|---|
| Paenibacillus sp. strain NRRL | 0.625 | 1 | 1 |
| B-50972 BC | 0.312 | 0 | 0 |
| | 0.156 | 1 | 1 |
| Paenibacillus sp. strain NRRL | 0.625 | 89 | 3 |
| B-50972 BC + 2% Urea | 0.312 | 79 | 0 |
| | 0.156 | 30 | 5 |
| Paenibacillus sp. strain NRRL | 0.625 | 88 | 2 |
| B-67304 BC + 2% Urea | 0.312 | 82 | 2 |
| | 0.156 | 38 | 11 |
| Paenibacillus sp. strain NRRL | 0.625 | 86 | 2 |
| B-67306 BC + 2% Urea | 0.312 | 80 | 8 |
| | 0.156 | 52 | 5 |

TABLE 14
Antifungal activity of broth concentrate (BC) from various
Paenibacillus sp. strains stabilized with urea against
Alternaria solani after one year of storage at 23° C.

| | | | |
|---|---|---|---|
| | | Average | |
| | Concentration | Efficacy | Standard |
| Treatment | (%) | (%) | Error |

Antifungal Activity of Controls

| | | | |
|---|---|---|---|
| Untreated Control | | 0 | — |
| SERENADE ® ASO (Bacillus | 5 | 91 | 5 |
| subtilis QST713) | 2.5 | 88 | 3 |
| | 1.25 | 80 | 5 |

Antifungal Activity after Storage at 23° C. for 1 Year

| | | | |
|---|---|---|---|
| Paenibacillus sp. strain NRRL | 0.625 | 30 | 8 |
| B-50972 BC | 0.312 | 11 | 6 |
| | 0.156 | 13 | 7 |
| Paenibacillus sp. strain NRRL | 0.625 | 86 | 2 |
| B-50972 BC + 2% Urea | 0.312 | 70 | 9 |
| | 0.156 | 42 | 14 |
| Paenibacillus sp. strain NRRL | 0.625 | 82 | 7 |
| B-67304 BC + 2% Urea | 0.312 | 74 | 4 |
| | 0.156 | 58 | 19 |
| Paenibacillus sp. strain NRRL | 0.625 | 82 | 9 |
| B-67306 BC + 2% Urea | 0.312 | 66 | 2 |
| | 0.156 | 20 | 9 |

### Example 9. Relative Fusaricidin a Levels and Antifungal Activity from Stabilized Formulations with Paenibacillus sp. Strains NRRL B-67304, NRRL B-67306, and NRRL B-67615

Whole broths and broth concentrates of Paenibacillus sp. NRRL B-67304, Paenibacillus sp. strain NRRL B-67306, and Paenibacillus sp. strain NRRL B-67615 were prepared as described in Example 1. No urea was added to the whole broths while the broth concentrates were stabilized with urea. The antifungal activities of the whole broths and stabilized broth concentrates against Botrytis cinerea and Alternaria solani were measured as described in Examples 1 and 3 after eight weeks of storage at 40° C. and after one year of storage at 23° C. In the antifungal assays, the whole broth samples were applied at twice the rates as those of the broth concentrate samples to adjust for the dilution of antifungal chemistries in the whole broth. The concentrations (i.e., application rates) are dilutions of the referenced treatment in water. 0% means an efficacy which corresponds to that of the untreated control, while an efficacy of 100% means that no disease is observed. Relative levels of fusaricidin A in the whole broths and broth concentrates were also determined as outlined in Example 2.

The antifungal activities of the whole broth samples of the three Paenibacillus sp. strains without urea were similar to or slightly greater than that of untreated control. In contrast, the antifungal activities of the urea-stabilized broth concentrates of the three Paenibacillus sp. strains remained relatively high and were more similar to that of the positive control, SERENADE® ASO (Bacillus subtilis QST713). This positive control was not subjected to the storage conditions of the stability assays and was known to have a relatively high antifungal actity. The stabilizing effect of the urea was consistent with both fungal pathogens (i.e., Botrytis cinerea and Alternaria solani) and over both periods (i.e., eight weeks of storage at 40° C. and one year of storage at 23° C.) (see Tables 15-18).

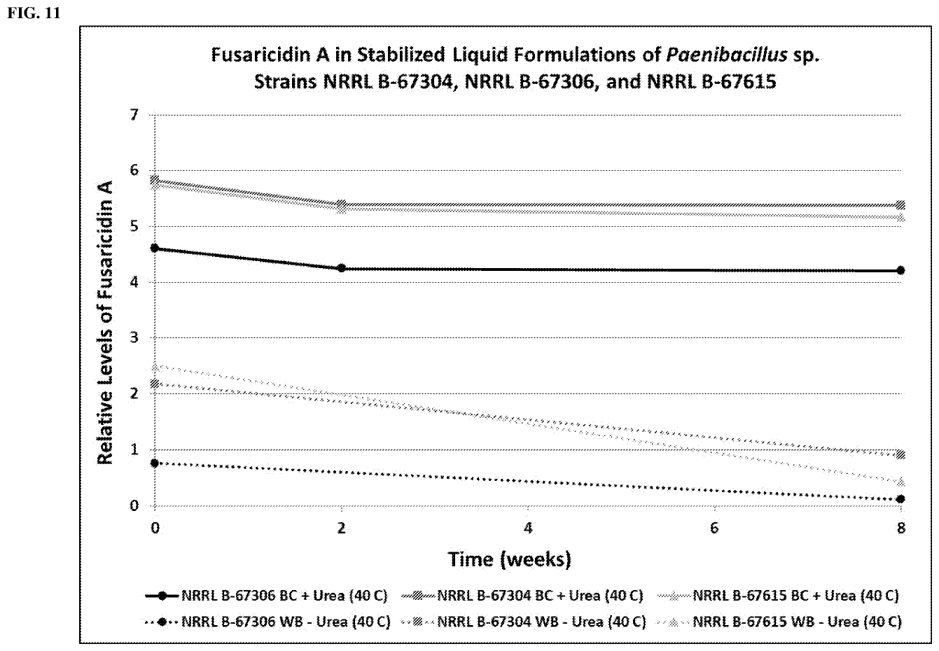

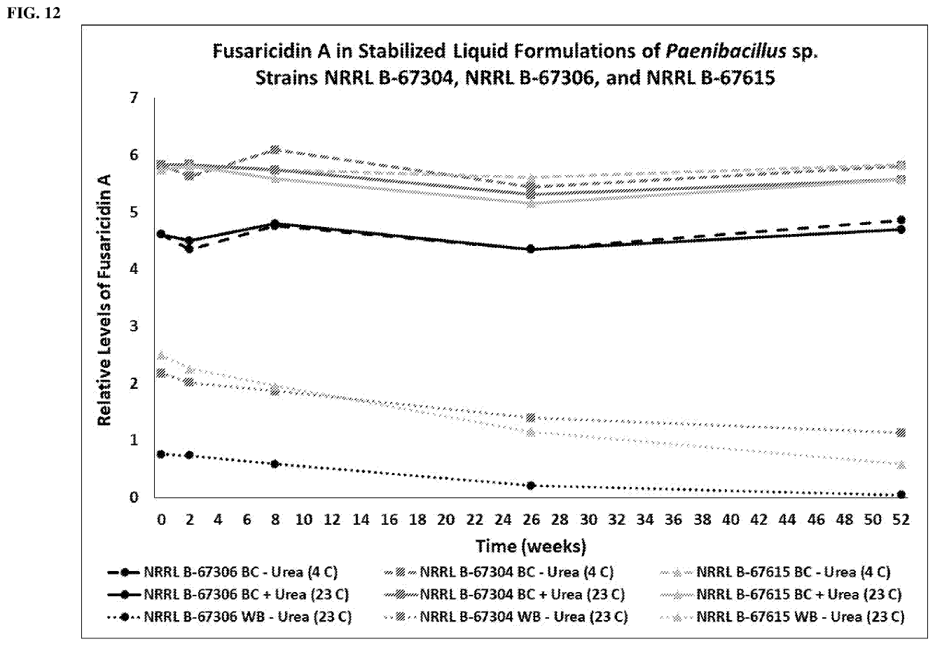

Fusaricidin A levels in the unstabilized whole broth samples and in the urea-stabilized broth concentrate samples of Paenibacillus sp. NRRL B-67304, Paenibacillus sp. strain NRRL B-67306, and Paenibacillus sp. strain NRRL B-67615 over eight weeks of storage at 40° C. and over one year of storage at 23° C. are presented in FIG. 11 and FIG. 12, respectively. While fusaricidin A levels in the unstabilized whole broth samples dropped significantly over these periods, those in the broth concentrate samples containing urea remained relatively constant.

TABLE 15
Antifungal activity of whole broth (WB) and broth concentrate
(BC) from Paenibacillus sp. strains NRRL B-67304, NRRL
B-67306, and NRRL B-67615 stabilized with urea against Botrytis cinerea
after eight weeks of storage at 40° C.

| | | | |
|---|---|---|---|
| | | Average | |
| | Concentration | Efficacy | Standard |
| Treatment | (%) | (%) | Error |

Antifungal Activity of Controls

| | | | |
|---|---|---|---|
| Untreated Control | | 0 | — |
| SERENADE ® ASO (Bacillus | 2.5 | 94 | 3 |
| subtilis QST713) | 1.25 | 9 | 6 |

Antifungal Activity after Storage at 40° C. for 8 Weeks

| | | | |
|---|---|---|---|
| Paenibacillus sp. strain NRRL | 1.25 | 33 | 12 |
| B-67304 WB | 0.313 | 5 | 5 |
| Paenibacillus sp. strain NRRL | 0.625 | 83 | 6 |
| B-67304 BC + 2% Urea | 0.156 | 48 | 14 |
| Paenibacillus sp. strain NRRL | 1.25 | 0 | 0 |
| B-67306 WB | 0.313 | 0 | 0 |
| Paenibacillus sp. strain NRRL | 0.625 | 74 | 5 |
| B-67306 BC + 2% Urea | 0.156 | 14 | 5 |
| Paenibacillus sp. strain NRRL | 1.25 | 4 | 4 |
| B-67615 WB | 0.313 | 0 | 0 |
| Paenibacillus sp. strain NRRL | 0.625 | 90 | 2 |
| B-67615 BC + 2% Urea | 0.156 | 45 | 7 |

TABLE 16
Antifungal activity of WB and BC from Paenibacillus
sp. strains NRRL B-67304, NRRL B-67306, and NRRL
B-67615 stabilized with urea against Botrytis cinerea
after one year of storage at 23° C.

| | | | |
|---|---|---|---|
| | | Average | |
| | Concentration | Efficacy | Standard |
| Treatment | (%) | (%) | Error |

Antifungal Activity of Controls

| | | | |
|---|---|---|---|
| Untreated Control | | 0 | — |
| SERENADE ® ASO (Bacillus | 2.5 | 95 | 0 |
| subtilis QST713) | 1.25 | 88 | 3 |

Antifungal Activity after Storage at 23° C. for 1 Year

| | | | |
|---|---|---|---|
| Paenibacillus sp. strain NRRL | 1.25 | 38 | 11 |
| B-67304 WB | 0.313 | 1 | 1 |
| Paenibacillus sp. strain NRRL | 0.625 | 63 | 1 |
| B-67304 BC + 2% Urea | 0.156 | 56 | 4 |
| Paenibacillus sp. strain NRRL | 1.25 | 1 | 1 |
| B-67306 WB | 0.313 | 0 | 0 |
| Paenibacillus sp. strain NRRL | 0.625 | 29 | 3 |
| B-67306 BC + 2% Urea | 0.156 | 23 | 14 |
| Paenibacillus sp. strain NRRL | 1.25 | 1 | 1 |
| B-67615 WB | 0.313 | 0 | 0 |
| Paenibacillus sp. strain NRRL | 0.625 | 50 | 12 |
| B-67615 BC + 2% Urea | 0.156 | 43 | 7 |

TABLE 17
Antifungal activity of WB and BC from Paenibacillus
sp. strains NRRL B-67304, NRRL B-67306, and NRRL
B-67615 stabilized with urea against Alternaria solani
after eight weeks of storage at 40° C.

| | | | |
|---|---|---|---|
| | | Average | |
| | Concentration | Efficacy | Standard |
| Treatment | (%) | (%) | Error |

Antifungal Activity of Controls

| | | | |
|---|---|---|---|
| Untreated Control | | 0 | — |
| SERENADE ® ASO (Bacillus | 2.5 | 90 | 4 |
| subtilis QST713) | 1.25 | 86 | 5 |

Antifungal Activity after Storage at 40° C. for 8 Weeks

| | | | |
|---|---|---|---|
| Paenibacillus sp. strain NRRL | 1.25 | 40 | 20 |
| B-67304 WB | 0.313 | 18 | 14 |
| Paenibacillus sp. strain NRRL | 0.625 | 84 | 8 |
| B-67304 BC + 2% Urea | 0.156 | 52 | 6 |
| Paenibacillus sp. strain NRRL | 1.25 | 3 | 3 |
| B-67306 WB | 0.313 | 0 | 0 |
| Paenibacillus sp. strain NRRL | 0.625 | 88 | 2 |
| B-67306 BC + 2% Urea | 0.156 | 47 | 25 |
| Paenibacillus sp. strain NRRL | 1.25 | 27 | 18 |
| B-67615 WB | 0.313 | 8 | 8 |
| Paenibacillus sp. strain NRRL | 0.625 | 71 | 9 |
| B-67615 BC + 2% Urea | 0.156 | 68 | 9 |

TABLE 18
Antifungal activity of WB and BC from Paenibacillus
sp. strains NRRL B-67304, NRRL B-67306, and NRRL
B-67615 stabilized with urea against Alternaria solani
after one year of storage at 23° C.

| | | | |
|---|---|---|---|
| | | Average | |
| | Concentration | Efficacy | Standard |
| Treatment | (%) | (%) | Error |

Antifungal Activity of Controls

| | | | |
|---|---|---|---|
| Untreated Control | | 0 | — |
| SERENADE ® ASO (Bacillus | 2.5 | 88 | 2 |
| subtilis QST713) | 1.25 | 82 | 2 |

Antifungal Activity after Storage at 23° C. for 1 Year

| | | | |
|---|---|---|---|
| Paenibacillus sp. strain NRRL | 1.25 | 18 | 9 |
| B-67304 WB | 0.313 | 24 | 14 |
| Paenibacillus sp. strain NRRL | 0.625 | 88 | 4 |
| B-67304 BC + 2% Urea | 0.156 | 65 | 18 |
| Paenibacillus sp. strain NRRL | 1.25 | 0 | 0 |
| B-67306 WB | 0.313 | 8 | 4 |
| Paenibacillus sp. strain NRRL | 0.625 | 82 | 8 |
| B-67306 BC + 2% Urea | 0.156 | 50 | 3 |
| Paenibacillus sp. strain NRRL | 1.25 | 7 | 3 |
| B-67615 WB | 0.313 | 0 | 0 |
| Paenibacillus sp. strain NRRL | 0.625 | 92 | 2 |
| B-67615 BC + 2% Urea | 0.156 | 72 | 7 |

Unless defined otherwise, all technical and scientific terms herein have the same meaning as commonly understood by one of ordinary skill in the art to which this invention belongs. All publications, patents, and patent publications cited are incorporated by reference herein in their entirety for all purposes.

It is understood that the disclosed invention is not limited to the particular methodology, protocols and materials described as these can vary. It is also understood that the terminology used herein is for the purposes of describing particular embodiments only and is not intended to limit the scope of the present invention which will be limited only by the appended claims.

Those skilled in the art will recognize, or be able to ascertain using no more than routine experimentation, many equivalents to the specific embodiments of the invention described herein. Such equivalents are intended to be encompassed by the following claims.

1. A stabilized fungicidal composition comprising:
a) a fusaricidin-producing Paenibacillus sp. strain; and
b) a stabilizing agent selected from the group consisting of an amine, a quaternary ammonium compound, a salt of phosphate or citrate, a short chain polyol having from 2 to 10 carbon atoms, a urea, and combinations thereof.

2-22. (canceled)

23. An agriculturally acceptable stable aqueous formulation comprising:
a) a fusaricidin-producing Paenibacillus sp. strain in an amount of 3% w/w to 90% w/w;
b) a stabilizing agent selected from the group consisting of an amine, a quaternary ammonium compound, a salt of phosphate or citrate, a short chain polyol having from 2 to 10 carbon atoms, a urea, and combinations thereof in an amount of 0.1% w/w to 20% w/w;
c) water; and
d) optionally, a polar water miscible organic solvent in an amount of 2% w/w to 60% w/w.

24. The aqueous formulation of claim 23, wherein the fusaricidin-producing Paenibacillus sp. strain is Paenibacillus sp. strain NRRL B-50972, Paenibacillus sp. strain NRRL B-67129, Paenibacillus sp. strain NRRL B-67304, Paenibacillus sp. strain NRRL B-67306, Paenibacillus sp. strain NRRL B-67615, or a fungicidal mutant strain thereof.

25. The aqueous formulation of claim 23, wherein the stabilizing agent is a urea.

26. The aqueous formulation of claim 25, wherein the urea is carbonic diamide (CO(NH₂)₂) or a substituted urea of formula (I) 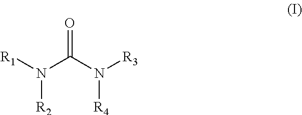
wherein
R₁, R₂, and R₃ are independently —H, —OH, C₁-C₆ alkyl, or —R₅OH;
R₄ is —R₅OH; and
R₅ is C₁-C₆ alkyl.

27. The aqueous formulation according to claim 25, further comprising a salt of phosphate or citrate.

28. The aqueous formulation according to claim 27, wherein the salt of phosphate or citrate is selected from the group consisting of potassium phosphate monobasic, potassium phosphate dibasic, potassium citrate monobasic, potassium citrate dibasic, and combinations thereof.

29. The aqueous formulation according to claim 27, wherein the urea and the salt of phosphate or citrate are present in the aqueous formulation at a combined concentration of between about 0.1% and about 3%.

30. The aqueous formulation of claim 23, wherein the stabilizing agent is an amine.

31. The aqueous formulation of claim 30, wherein the amine is guanidine hydrochloride or triethanolamine.

32. The aqueous formulation of claim 23, wherein the stabilizing agent is a quaternary ammonium compound.

33. The aqueous formulation of claim 32, wherein the quaternary ammonium compound is betaine hydrochloride.

34. The aqueous formulation of claim 23, wherein the stabilizing agent is a salt of phosphate or citrate.

35. The aqueous formulation of claim 34, wherein the salt is potassium phosphate (dibasic) or potassium citrate (tribasic).

36. The aqueous formulation of claim 23, wherein the stabilizing agent is a short chain polyol having from 2 to 10 carbon atoms.

37. The aqueous formulation of claim 36, wherein the short chain polyol having from 2 to 10 carbon atoms is selected from the group consisting of diethylene glycol, triethylene glycol, propylene glycol, 1,3-butylene glycol, tetramethylene glycol, pentamethylene glycol, hexamethylene glycol, ethylene glycol, 1,3-propylene glycol, 1,4-butanediol, neopentyl glycol, 3-methyl-1,5-pentanediol, p-xylene glycol, 1,4-bis-(β-hydroxyethoxy)benzene, 1,3-bis-(β-hydroxyethoxy)benzene, cyclohexane 1,4-dimethanol, octane-1,8-diol, decane-1,10-diol, and combinations thereof.

38. The aqueous formulation of claim 37, wherein the short chain polyol having from 2 to 10 carbon atoms is propylene glycol.

39. A method of treating a plant to control a disease, wherein the method comprises applying an effective amount of an aqueous formulation according to claim 23 to the plant, to a part of the plant and/or to a locus of the plant.

40. The method of claim 39, wherein the method comprises applying the fungicidal composition or aqueous formulation to foliar plant parts.

41. The method of claim 39, wherein the disease is caused by a fungal pathogen selected from the group consisting of Alternaria alternata, Alternaria solani, Botrytis cinerea, Colletotrichum lagenarium, Fusarium culmorum, Phaeosphaeria nodorum, Zymoseptoria tritici, Phytophthora cryptogea, Phytophthora infestans, Pseudoperonospora cubensis, Pythium ultimum, Magnaporthe oryzae, Thanatephorus cucumeris, Ustilago segetum var. avenae, Uromyces appendiculatus, and Puccinia triticina.
